(19) United States
(12) Patent Application Publication
(10) Pub. No.: US 2018/0168106 A1
(43) Pub. Date: Jun. 21, 2018

(54) METHODS FOR PROVIDING GRAFTED CALIBRACHOA AND PRODUCTS THEREOF

(71) Applicant: HISHTIL LTD., MOSHAV NEHALIM (IL)

(72) Inventors: Amit DAGAN, MOSHAV MAZOR (IL); Menachem SHADMI, YAVNE (IL); Alon PERRI, MOSHAV MAZOR (IL)

(73) Assignee: HISHTIL LTD., MOSHAV NEHALIM (IL)

(21) Appl. No.: 15/509,868

(22) PCT Filed: Sep. 9, 2015
(86) PCT No.: PCT/IL2015/050922

§ 371 Date: Mar. 9, 2017

Related U.S. Application Data
(60) Provisional application No. 62/048,814, filed on Sep. 11, 2014.

(51) Int. Cl. A01G 2/30; A01H 5/02; A01G 22/60; A01H 1/04

(52) U.S. Cl. CPC A01G 2/30; A01H 1/04; A01G 22/60; A01H 5/02

(57) ABSTRACT

The present invention discloses a grafted Calibrachoa plant. The grafted plant comprises a selected rootstock engrafted with a Calibrachoa scion. The present invention further provides novel grafted propagation material and methods for producing the grafted propagation material and grafted Calibrachoa plants.

### FIELD OF THE INVENTION

The present invention generally relates to the field of grafted plants as well as to a method for providing grafted plants.

### BACKGROUND OF THE INVENTION

Calibrachoa is a genus of plants in the Solanaceae (nightshade) family. These plants are weak, evergreen, short-lived perennials and subshrubs, with a sprawling growth pattern. The Calibrachoa plant has small Petunia-type flowers.

Calibrachoa are closely related to Petunia. However it has been found that there are major differences in chromosomes, corresponding to external differences and fertilization factors that distinguished the two genera. With regard to Calibrachoa, there appears to be little taxonomic information. The publication available on http://www.clamerinforma.it/News/NewsDetEn.asp?id+1480 describes the leaf anatomy of sixteen species of Calibrachoa and eight species of Petunia to separate the two genera and to characterize the various Calibrachoa species. Both Calibrachoa and Petunia genera are important as ornamental, medicinal and nutritious species. Petunia has a chromosomal ploidy level of n=7. Most species are herbaceous and annual. Calibrachoa species are predominantly sub-arbustive and perennial, and ornamental varieties are derived essentially from Calibrachoa parviflora. It was further reported that a large number of Calibrachoa species demonstrate considerable variation, especially in vegetative characteristics.

Plant breeders have created varieties by the hybridization of Calibrachoa and Petunia species and selection programs. For example, Petchoa is a hybrid genus derived from crossing the genetically similar Calibrachoa and Petunia. Moreover, garden hybrids originate from crosses between Petunia axillaris and Petunia integrifolia.

U.S. Pat. No. 7,891,134 “METHOD OF PRODUCING A HORTICULTURAL DISPLAY” to Wismans, discloses a method of producing a horticultural display containing more than one variety of the same or different species of plant. The plants are produced from unrooted cuttings that are rooted in a single growth cell and cultivated in a single container so that the final product includes more than one plant species or more than one plant variety or both. It is further disclosed that the unrooted plant cuttings may include species of Petunia and Calibrachoa. The species selections are based on the attractive complementary colors and matching growth habits and flowering times. However, since the Petunia species and Calibrachoa species have distinct plant vigor and rooting properties, significant adjustment effort is required to obtain the two plant species or plant varieties merely grown in the same container.

U.S. Pat. No. 7,786,342 “DOUBLE FLOWER CALIBRACHOA BREEDING METHODS AND PLANTS PRODUCED THEREFROM” to Stover, discloses a method for breeding double flower Calibrachoa sp. plants by crossing two parent plants of the genus Calibrachoa.

US patent application 20140059710A1 “CALIBRACHOA PLANT WITH STAR PATTERN” to Sakazaki, teaches Calibrachoa plants displaying at least one inflorescence having dark yellow colored flowers with a white-colored star pattern. The Calibrachoa plants are produced by crossing, a first Calibrachoa plant comprising at least one inflorescence with the star-shaped pattern, with a second Calibrachoa plant that does not comprise an inflorescence with the star-shaped pattern, and selecting progeny that have the star-shaped pattern.

In view of the above, it is apparent that there is a long felt and unmet need for producing engrafted Calibrachoa plants having improved agronomical and ornamental properties that are highly desired commercially.

### SUMMARY OF THE INVENTION

It is therefore one object of the present invention to disclose a grafted Calibrachoa plant, wherein said plant comprises a rootstock engrafted with a Calibrachoa scion.

It is a further object of the present invention to disclose the plant as defined above, wherein said rootstock is compatible with said Calibrachoa scion.

It is a further object of the present invention to disclose the plant as defined in any of the above, wherein said plant is derived from propagation material selected from the group consisting of young plants and unrooted raw material.

It is a further object of the present invention to disclose the plant as defined in any of the above, wherein said young plants are selected from the group consisting of rooted cuttings, seedlings, grafted rooted cuttings, and grafted seedlings.

It is a further object of the present invention to disclose the plant as defined in any of the above, wherein said unrooted raw material is selected from the group consisting of unrooted cuttings and unrooted grafted cuttings.

It is a further object of the present invention to disclose the plant as defined in any of the above, wherein said unrooted raw material is adapted for insertion and growth in plug trays.

It is a further object of the present invention to disclose a finished plant derived from the propagation material as defined in any of the above.

It is a further object of the present invention to disclose the plant as defined in any of the above, wherein said rootstock is derived from propagation material selected from the group consisting of seeds, rooted cuttings, unrooted cuttings, seedlings, grafted rooted cuttings, grafted unrooted cuttings and grafted unrooted seedlings.

It is a further object of the present invention to disclose the plant as defined in any of the above, wherein said rootstock belongs to the Solanaceae family.

It is a further object of the present invention to disclose the plant as defined in any of the above, wherein said rootstock is selected from the group consisting of Cestroideae, Goetzeoideae, Nicotianoideae, Petunioideae, Schizanthoideae, Schwenckioideae and Solanoideae subfamily.

It is a further object of the present invention to disclose the plant as defined in any of the above, wherein said rootstock is selected from the group consisting of Solanum, Capsicum, Petunia and Calibrachoa genus.

It is a further object of the present invention to disclose the plant as defined in any of the above, wherein said rootstock is selected from the group consisting of Petunia alpicola, P. altiplana, P. axillaris, P. bajeensis, P. bonjardinensis, P. exserta, P. guarapuavensis, P. helianthemoides, P. humifusa, P. inflate, P. integrifolia, P. interior, P. ledifolia, P. littoralis, P. mantiqueirensis, P. occidentalis, P. parviflora, P. patagonica, P. pubescens, P. reitzii, P. riograndensis, P. saxicola, P. scheideana, P. variabilis, P. villadiana, P.×atkinsiana and P. hybrida species.

It is a further object of the present invention to disclose the plant as defined in any of the above, wherein said rootstock is selected from the group consisting of Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia, Calibrachoa×hybrida and Petchoa species.

It is a further object of the present invention to disclose the plant as defined in any of the above, wherein said rootstock is selected from the group consisting of lycopersicum, tuberosum and melongena species.

It is a further object of the present invention to disclose the plant as defined in any of the above, wherein said scion is derived from propagation material selected from the group consisting of seeds, rooted cuttings, unrooted cuttings, seedlings, grafted rooted cuttings, grafted unrooted cuttings and grafted unrooted seedlings.

It is a further object of the present invention to disclose the plant as defined in any of the above, wherein said scion is of the Calibrachoa genus.

It is a further object of the present invention to disclose the plant as defined in any of the above, wherein said scion is selected from the group consisting of Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia species.

It is a further object of the present invention to disclose the plant as defined in any of the above, wherein said rootstock has at least one compatible characteristic with said scion.

It is a further object of the present invention to disclose the plant as defined in any of the above, wherein said at least one compatible characteristic with said scion is selected from the group consisting of: similar stem or shoot diameter, similar growth pattern, taxonomical similarity, genetic similarity, anatomical similarity, formation of uniform adhesion point between said rootstock and said scion, absence of noticeable callus, suppressed formation of side shoots from the stem, suppressed formation of sprouts from the callus, a thick and woody stem, normal development of said scion similar or enhanced relative to a corresponding none grafted control plant, and any combination thereof.

It is a further object of the present invention to disclose the plant as defined in any of the above, wherein said rootstock has desirable agronomical and/or horticultural characteristics.

It is a further object of the present invention to disclose the plant as defined in any of the above, wherein said desirable agronomical and/or horticultural characteristics are selected from the group consisting of high rooting potential, high rooting quality, developed root system, callus formation, minimal sprouts from the callus, minimal side-shoots development from the stem, thick stem, long internodes, robustness, sparse flowering, production of a straight trunk, woodiness, strong root system, enhanced stem height, plant vigor, stress tolerance, disease resistance, resistance to pathogens, resistance to insect infestation, resistance to abiotic stress and any combination thereof.

It is a further object of the present invention to disclose the plant as defined in any of the above, wherein said desirable agronomical and/or horticultural characteristic is selected from the group consisting of a rooting potential of at least 80%, sprout differentiation from callus lower than 80%, side shoots from the stem lower than 90%, average stem thickness of at least 5 mm, average internode length of at least 10 mm and any combination thereof.

It is a further object of the present invention to disclose the plant as defined in any of the above, wherein the stress tolerance comprises an abiotic stress tolerance selected from the group consisting of cold tolerance, high temperature tolerance, drought tolerance, salt tolerance, pH tolerance and any combination thereof.

It is a further object of the present invention to disclose the plant as defined in any of the above, wherein the stress tolerance comprises a biotic stress tolerance selected from the group consisting of a disease resistance, an insect resistance, and a nematode resistance, improved resistance to soil borne pathogens and any combination thereof.

It is a further object of the present invention to disclose the plant as defined in any of the above, wherein said scion is engrafted at a height of up to 50 cm on said rootstock.

It is a further object of the present invention to disclose the plant as defined in any of the above, wherein said scion is engrafted at a height of at least 10 cm on said rootstock.

It is a further object of the present invention to disclose the plant as defined in any of the above, wherein said scion is characterized by at least one characteristic selected from the group consisting of dense flowering, unique flower color, low to moderate incompatibility with the rootstock, at least 50% output of developed plants, and any combination thereof.

It is a further object of the present invention to disclose the plant as defined in any of the above, wherein said rootstock is engrafted with at least two different scions said scions belong to different varieties and are compatible with said rootstock.

It is a further object of the present invention to disclose the plant as defined in any of the above, wherein said different scions have at least one different characteristic selected from the group consisting of: inflorescence color, fruit shape, growth pattern and any combination thereof.

It is a further object of the present invention to disclose the plant as defined in any of the above, wherein said engrafted Calibrachoa plant has desirable ornamental and/or agronomical and/or horticultural characteristics relative to a non-grafted Calibrachoa plant selected from the group consisting of: enhanced height or high stem plant, uniform adhesion point, woodiness, similar shoot diameter between the rootstock and the scion, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof.

It is a further object of the present invention to disclose the plant as defined in any of the above, wherein said plant is planted into a final commercial planter or pot or any other growing container.

It is a further object of the present invention to disclose the plant as defined in any of the above, wherein said final commercial planter or pot or any other growing container has a volume of 1 liter and less.

It is a further object of the present invention to disclose the plant as defined in any of the above, wherein said final commercial planter or pot or any other growing container has a volume of 1 liter and more.

It is a further object of the present invention to disclose the plant as defined in any of the above, wherein said plant is a high stem grafted Calibrachoa.

It is a further object of the present invention to disclose the plant as defined in any of the above, wherein said high stem grafted Calibrachoa plant is derived from propagation material selected from the group consisting of high stem grafted young plants and unrooted high stem grafted raw material.

It is a further object of the present invention to disclose the plant as defined in any of the above, wherein said high stem young plants are selected from the group consisting of rooted cuttings, seedlings, grafted rooted cuttings, and grafted seedlings.

It is a further object of the present invention to disclose the plant as defined in any of the above, wherein said high stem unrooted raw material is selected from the group consisting of unrooted cuttings and unrooted grafted cuttings.

It is a further object of the present invention to disclose the plant as defined in any of the above, wherein said high stem unrooted raw material is adapted for perpendicular stability and/or sturdiness when planted in a plug tray.

It is a further object of the present invention to disclose a high stem grafted finished plant derived from the propagation material as defined in any of the above.

It is a further object of the present invention to disclose the finished plant as defined in any of the above, wherein said plant is adapted for perpendicular stability and/or sturdiness when planted in a pot or planting container.

The cuttings or unrooted cuttings, grafted cuttings or grafted unrooted cuttings of the high stem grafted Calibrachoa as defined in any of the above.

It is a further object of the present invention to disclose any part of the plant as defined in any of the above.

It is a further object of the present invention to disclose the plant as defined in any of the above, wherein said plant part is selected from the group consisting of rooted cuttings, unrooted cuttings, grafted rooted cuttings, unrooted grafted cuttings, flowers and any other vegetative or reproductive part of the plant.

It is a further object of the present invention to disclose grafted propagation material of the plant as defined in any of the above, wherein said propagation material is selected from the group consisting of grafted young plants, grafted rooted cuttings, grafted unrooted cuttings, grafted seedlings, high stem grafted young plants and high stem grafted unrooted cuttings.

It is a further object of the present invention to disclose a method for producing a Calibrachoa engrafted plant comprising steps of: (a) providing a scion of the genus Calibrachoa; (b) conditioning a rootstock for compatibility with said Calibrachoa scion; and (c) grafting said scion onto said rootstock, thereby producing a grafted Calibrachoa plant.

It is a further object of the present invention to disclose the method as defined above, wherein said step of conditioning, further comprises steps of growing said rootstock to have desirable agronomical and/or horticultural characteristics.

It is a further object of the present invention to disclose the method as defined in any of the above, wherein said desirable agronomical and/or horticultural characteristics are selected from the group consisting of high rooting potential, high rooting quality, minimal side-shoots development from the stem, thick stem, long internodes, robustness, sparse flowering, producing a straight trunk, woodiness, strong root system, enhanced stem height, plant vigor, stress tolerance, disease resistance, resistance to pathogens, resistance to insect infestation, resistance to abiotic stress and any combination thereof.

It is a further object of the present invention to disclose the method as defined in any of the above, wherein said desirable agronomical and/or horticultural characteristic is selected from the group consisting of a rooting potential of at least 80%, sprout differentiation from callus lower than 80%, side shoots from the stem lower than 90%, average stem thickness of at least 5 mm, average internodes elongation of at least 10 mm and any combination thereof.

It is a further object of the present invention to disclose the method as defined in any of the above, further comprises steps of selecting a scion having at least one characteristic selected from the group consisting of dense flowering, unique flower colors, low to moderate incompatibility with the rootstock, at least 50% output of developed plants, and any combination thereof.

It is a further object of the present invention to disclose the method as defined in any of the above, wherein said step of conditioning further comprises steps of growing said rootstock grafted upon said scion and obtaining characteristics selected from the group consisting of a uniform adhesion point between said rootstock and said scion, similar shoot diameters between said rootstock and said scion, absence of noticeable callus, suppressed formation of side shoots from the stem, suppressed formation of sprouts from the callus and any combination thereof.

It is a further object of the present invention to disclose the method as defined in any of the above, further comprises steps of producing and growing propagation material selected from the group consisting of young plants and unrooted raw material at predetermined growth conditions so as to provide said Calibrachoa grafted plant.

It is a further object of the present invention to disclose the method as defined in any of the above, further comprises steps of selecting said young plants from the group consisting of rooted cuttings, seedlings, grafted rooted cuttings, and grafted seedlings.

It is a further object of the present invention to disclose the method as defined in any of the above, further comprises steps of selecting said unrooted raw material from the group consisting of unrooted cuttings and unrooted grafted cuttings.

It is a further object of the present invention to disclose the method as defined in any of the above, further comprises steps of growing said unrooted raw material in plug trays.

It is a further object of the present invention to disclose the method as defined in any of the above, further comprises steps of growing said propagation material so as to produce a finished grafted Calibrachoa plant.

It is a further object of the present invention to disclose the method as defined in any of the above, further comprises steps of growing propagation material or young plants or raw material selected from the group consisting of seeds, rooted cuttings, unrooted cuttings, seedlings, grafted rooted cuttings, grafted unrooted cuttings and grafted seedlings of said rootstock plant at predetermined growth conditions so as to be suitable for grafting with said scion.

It is a further object of the present invention to disclose the method as defined in any of the above, further comprises steps of growing propagation material or young plants or raw material selected from the group consisting of seeds, rooted cuttings, unrooted cuttings, seedlings, grafted rooted cuttings, grafted unrooted cuttings and grafted seedlings of said scion at predetermined growth conditions so as to be suitable for grafting with said rootstock.

It is a further object of the present invention to disclose the method as defined in any of the above, comprising additional steps of adjusting the density of the grown rootstock plants by controlling the number of plants per area.

It is a further object of the present invention to disclose the method as defined in any of the above, comprising additional steps of selecting a suitable growth tray by parameters selected from the group consisting of size, shape of cells, material, density and any combination thereof.

It is a further object of the present invention to disclose the method as defined in any of the above, comprising additional steps of growing said rootstock under predetermined fertigation and shading regimes.

It is a further object of the present invention to disclose the method as defined in any of the above, comprising additional steps of applying controlled Low-Radiation regimes for obtaining etiolated rootstock plants.

It is a further object of the present invention to disclose the method as defined in any of the above, comprising additional steps of controlling the flowering timing of the grown rootstock plants by affecting photoperiodic response of the plants.

It is a further object of the present invention to disclose the method as defined in any of the above, comprising additional steps of using trailing and stabilizing means for supporting and shaping the grown rootstock.

It is a further object of the present invention to disclose the method as defined in any of the above, comprising additional steps of using trailing and stabilizing means selected from the group consisting of sticks, clips, flowers cutting, trailing net, plastic tray frame and any combination thereof.

It is a further object of the present invention to disclose the method as defined in any of the above, comprising additional steps of growing the selected rootstock plants at a predetermined protocol by controlling parameters selected from the group consisting of temperature, relative humidity, electrical conductivity, illuminating level, shading level, pH, irrigation intervals, fertilization solutions and regimes and any combination thereof.

It is a further object of the present invention to disclose the method as defined in any of the above, comprising additional steps of applying plant protection treatments selected from the group consisting of: pesticide-based treatment, herbicides treatment, insecticides treatment, fungicides treatment, biological pest control treatment and any combination thereof.

It is a further object of the present invention to disclose the method as defined in any of the above, comprising additional steps of coordinating at least one property of the plant propagation material to be grown for rootstock production, said at least one property is selected from the group consisting of root system development, including properties such as development rate, conditions for the development, size, functionality and rooting output; hypocotyl size; hypocotyl length; stem diameter; nodes number; internode length and any combination thereof.

It is a further object of the present invention to disclose the method as defined in any of the above, further comprises steps of enhancing adhesion between said scion and said rootstock to form a graft union.

It is a further object of the present invention to disclose the method as defined in any of the above, further comprises steps of growing said rootstock to a predetermined height of up to 50 cm and to a predetermined stem diameter.

It is a further object of the present invention to disclose the method as defined in any of the above, further comprises steps of growing said rootstock to a predetermined height of at least 10 cm.

It is a further object of the present invention to disclose the method as defined in any of the above, further comprises steps of manipulating and coordinating the growth of said rootstock plant so as to achieve growth characteristics suitable for grafting with said scion, said growth characteristics are selected from the group consisting of: root system development, hypocotyl size, hypocotyl length, stem diameter, nodes number, node length, stem diameter, stem height, growth pattern, flowering time and any combination thereof.

It is a further object of the present invention to disclose the method as defined in any of the above, further comprises steps of grafting said scion onto said rootstock by a grafting technique selected from the group consisting of: splice grafting, bud grafting, cleft grafting, side grafting, approach grafting, hole insertion grafting, one cotyledon grafting, whip grafting, stub grafting, four flap grafting, awl grafting, veneer grafting and by any other grafting technique suitable for grafting herbaceous plant and any combination thereof.

It is a further object of the present invention to disclose the method as defined in any of the above, further comprises steps of planting said grafted plant into a final commercial planter or pot or any other growing container.

It is a further object of the present invention to disclose the method as defined in any of the above, further comprises steps of planting said grafted plant into a final commercial planter or pot or any other growing container having a volume of 1 liter and less.

It is a further object of the present invention to disclose the method as defined in any of the above, further comprises steps of planting said grafted plant into a final commercial planter or pot or any other growing container having a volume of 1 liter and more.

It is a further object of the present invention to disclose the method as defined in any of the above, further comprises steps of producing an engrafted Calibrachoa plant with desirable ornamental and/or horticultural characteristics relative to non-grafted Calibrachoa plant selected from the group consisting of: enhanced height or high stem plant, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof.

It is a further object of the present invention to disclose the method as defined in any of the above, further comprises steps of producing a high stem grafted Calibrachoa.

It is a further object of the present invention to disclose the method as defined in any of the above, further comprises steps of growing the rootstock to a predetermined height of at least about 10 cm and predetermined stem diameter and grafting the Calibrachoa scion upon said rootstock at said predetermined height.

It is a further object of the present invention to disclose the method as defined in any of the above, further comprises steps of producing and growing propagation material selected from the group consisting of high stem young plants and unrooted high stem raw material at predetermined growth conditions so as to provide said Calibrachoa high stem grafted plant.

It is a further object of the present invention to disclose the method as defined in any of the above, further comprises steps of selecting said high stem young plants from the group consisting of rooted high stem cuttings, high stem seedlings, grafted high stem rooted cuttings, and grafted high stem seedlings.

It is a further object of the present invention to disclose the method as defined in any of the above, further comprises steps of selecting said high stem unrooted raw material from the group consisting of unrooted high stem cuttings and unrooted high stem grafted cuttings.

It is a further object of the present invention to disclose the method as defined in any of the above, further comprises steps of planting said high stem unrooted high stem raw material in plug trays.

It is a further object of the present invention to disclose the method as defined in any of the above, further comprises steps of growing said high stem unrooted high stem raw material with perpendicular stability and/or sturdiness in plug trays.

It is a further object of the present invention to disclose the method as defined in any of the above, further comprises steps of growing said propagation material so as to produce a finished high stem grafted Calibrachoa plant.

It is a further object of the present invention to disclose any part of a plant produced by the method as defined in any of the above.

It is a further object of the present invention to disclose the plant part as defined above, wherein said plant part is selected from the group consisting of rooted cuttings, unrooted cuttings, grafted rooted cuttings, unrooted grafted cuttings, flowers and any other vegetative or reproductive part of the plant.

It is a further object of the present invention to disclose grafted propagation material of a plant produced by the method as defined in any of the above, wherein said propagation material is selected from the group consisting of grafted young plants, grafted rooted cuttings, grafted unrooted cuttings, grafted seedlings, high stem grafted young plants and high stem grafted unrooted cuttings.

It is a further object of the present invention to disclose the method as defined in any of the above, further comprises steps of engrafting said rootstock with at least two different scions.

It is a further object of the present invention to disclose the method as defined in any of the above, further comprises steps of engrafting said rootstock with at least two different scions, said scions belong to different varieties and are compatible with said rootstock.

It is a further object of the present invention to disclose the method as defined in any of the above further comprises steps of providing said different scions having at least one different characteristic selected from the group consisting of: inflorescence color, fruit shape, growth pattern and any combination thereof.

### BRIEF DESCRIPTION OF THE DRAWINGS

In order to understand the invention and to see how it may be implemented in practice, several embodiments will now be described, by way of non-limiting example only, with reference to the accompanying drawing, in which:

FIG. 1A presents a photographic illustration demonstrating a high rooting potential of different Petunia surfinia rootstocks;

FIG. 1B presents a photographic illustration of a high stem Petunia surfinia rootstocks produced by methods inter alia described in the present invention;

FIG. 2 presents a photographic illustration of callus formation, rooting and the undesirable phenomena of sprouts growth from the callus of a Calibrachoa sp. grafted upon Petunia rootstocks;

FIG. 3 presents a photographic illustration demonstrating growth of sprouts from callus of a Calibrachoa sp. grafted upon Petunia rootstocks;

FIG. 4 presents a photographic illustration of trunk development and thickness of different Petunia rootstocks engrafted with a Calibrachoa scion;

FIG. 5 presents a photographic illustration demonstrating incompatibility between the rootstocks Petunia sp. ‘Crazytunia’ models Mandeville and Stone-was and Calibrachoa sp. ‘Chameleon’ models Pink and Purple scions;

FIG. 6 presents a photographic illustration of grafting point breakdown of Calibrachoa sp. Chameleon pink grafted upon Petunia surfinia pink;

FIG. 7 presents a photographic illustration of engrafted Calibrachoa plants of various inflorescence color;

FIG. 8 presents a photographic illustration of a shaped, and enhanced-height, engrafted Calibrachoa plant grown for two months under greenhouse conditions;

FIG. 9 presents a photographic illustration of a short-rootstock engrafted Calibrachoa plant;

FIG. 10 presents a photographic illustration of plants of the genus Petunia, grown as a rootstock, as an embodiment of the present invention;

FIG. 11 presents a photographic illustration of a Calibrachoa plant grafted upon Petunia rootstock at a height of about 20 cm;

FIG. 12 presents a photographic illustration of engrafted Calibrachoa plant before (FIG. 12A) after (FIG. 12B) pruning and shaping applications;

FIG. 13 presents a photographic illustration of a mature engrafted Calibrachoa plant after pruning;

FIG. 14 presents a photographic illustration of enhanced height Calibrachoa grafted plant, 2 to 3 weeks after transplantation to a pot;

FIG. 15 presents a photographic illustration of a Calibrachoa scion grafted upon rootstock of the Solanum genus as an embodiment of the present invention.

### DETAILED DESCRIPTION OF THE PREFERRED EMBODIMENTS

The following description is provided, alongside all chapters of the present invention, so that to enable any person skilled in the art to make use of the invention and sets forth the best modes contemplated by the inventor of carrying out this invention. Various modifications, however, will remain apparent to those skilled in the art, since the generic principles of the present invention have been defined specifically to provide means and methods for producing a grafted Calibrachoa plant and a product thereof.

The commercial need to combine the desirable ornamental characteristics of Calibrachoa, for example, the diversity of flower colors (e.g. dark, yellow, orange and red), along with vigorous growth characteristics (e.g. strong root system, sturdy stem, uniformity and/or tolerance to heat, salt and diseases) provided by engrafting the Calibrachoa with a suitable rootstock, is met and achieved by the present invention.

The Calibrachoa plant is highly desirable for its ornamental characteristics, especially, for the diversity of its inflorescence color. However, these plants have relatively weak root system and are rather susceptible to various soil and water qualities. The present invention discloses for the first time the grafting of a Calibrachoa scion upon a selected compatible rootstock with desirable horticultural and ornamental characteristics such as at least one of the following novel characteristics: high stem, strong root system, vigorous growth and tolerance to biotic and/or abiotic stresses. Thus a new and surprising commercially valuable Calibrachoa engrafted plant is provided.

The production and provision of the grafted Calibrachoa plants of the present invention enable for the first time, the achievement of unique and innovative ornamental and horticultural combinations, for example, enhanced height Calibrachoa plants as compared to the currently available shrubby-type Calibrachoa, grafted Calibrachoa plants with desirable added value such as heat, alkalinity and salt tolerance as compared to the currently available weak and sensitive varieties, uniquely designed engrafted Calibrachoa plants and new combinations of Calibrachoa species or varieties within one plant.

The present invention comprises the following novel and unexpected merits:
1. The creation and provision of grafted Calibrachoa plants;
2. The creation and provision of grafted Calibrachoa propagation material in the form of young plants, or in the form of unrooted raw material adapted for planting in plug trays;
3. The creation and provision of high stem grafted Calibrachoa;
4. The creation and provision of high stem grafted propagation material in the form of young plants or in the form of unrooted raw material adapted for planting in plug trays.

Furthermore, the present invention provides grafted Calibrachoa source plants or mother plants which produce a higher yield of cuttings and therefore increase the efficiency of the cutting yield.

In accordance with a one embodiment, the present invention provides novel grafted propagation material in the form of young plants or any other raw material which is used by growers to produce and develop the finished grafted Calibrachoa product and sell it on to retailers or end users.

It is within the scope of the present invention to provide propagation material such as young plants or unrooted raw material that is used to produce flowering pots of grafted Calibrachoa. Such propagation material encompass, for example, (1) young plants comprising rooted cuttings, seedlings, grafted rooted cuttings and grafted seedlings, and (2) unrooted raw material or propagation material comprising unrooted cuttings and grafted unrooted cuttings. The unrooted, grafted propagation material may be inserted into plug trays for the development of roots and thereafter transplanted by finishers into pots or planting containers for further growth and thereafter delivered to retailers to be sold on to the consumers.

Thus according to a further embodiment, a finished grafted plant is produced from the propagation material in the form of young plants or other forms of raw material and is provided by the present invention.

In a further embodiment, the present invention provides high stem grafted Calibrachoa, which may be grafted on cuttings or seedlings.

In further aspects, the high stem grafted Calibrachoa provided by the present invention is adapted for perpendicular stability and/or sturdiness when planted in a pot or planting containers. The high stem grafted Calibrachoa has desirable properties such as vigorous growth and strong stem and root system, which enable its sturdiness and perpendicular stability when planted in a pot.

In a further embodiment, high stem grafted propagation material in the form of young plants or in the form of unrooted raw material is provided by the present invention for the first time. The high stem grafted unrooted raw material, e.g. unrooted cuttings, are adapted for insertion and development of roots in plug trays. Once rooted, the high stem grafted young plants are ready for transplanting to pots or planting containers for finishing off.

Furthermore, the invention facilitates production and supply of unrooted grafted cuttings for the production of grafted Calibrachoa young plants.

It should be emphasized that unrooted grafted cuttings of various plants' genus are produced and sold, but no Calibrachoa unrooted grafted cuttings or high stem grafted (or not grafted) unrooted cuttings are exist in the prior art. Such Calibrachoa unrooted grafted cuttings or high stem grafted unrooted cuttings are, for the first time, surprisingly provided by the present invention and are capable of being embedded, planted in pots or rooted in plug trays, sturdily and/or perpendicularly stably grown in the plug trays and sold for on to finishers to be planted in pots, in growing containers or in flower beds.

It is further noted that there are grafted young plants, produced and sold in the prior art, but no Calibrachoa grafted young plants or high stem (grafted or not) young plants. Such Calibrachoa grafted young plants or high stem grafted young plants, are, for the first time, surprisingly provided by the present invention.

The aforementioned young plants can be produced from grafted or not, cuttings or seedlings and are sold or provided to finishers to be transplanted to pots or planting containers.

### Definitions

The term “engrafted plant” or “grafted plant” as used hereinafter generally refers to a plant comprising a rootstock and at least one scion, wherein the scion(s) is (are) grafted onto the rootstock by any method known in the art. According to certain embodiments, the scion is of the Calibrachoa genus. It is noted that, for a successful graft union, the vascular cambium of the stock and scion should be in close contact, and an intermingling callus from stock and scion is formed.

As used herein, the term “rootstock” as used hereinafter refers to part of a plant comprising the stem and/or underground part or rooting system of that plant and onto which a scion, cutting or bud is intended to be grafted. According to some embodiments it can refer to a rhizome or stem. The rootstock is generally selected for its characteristics including, interaction with the soil, providing the roots and the stem to support the new plant, obtaining water and minerals from the soil, and providing resistance or tolerance to the relevant pests, diseases and/or other stress such as heat and salt tolerance.

It is further within the scope that the rootstock may be chosen by a person skilled in the art for its rooting characteristics, its disease resistances and for the vigor it can confer to the engrafted plant. According to one embodiment, the rootstock of the plant of the present invention may be selected by its compatibility characteristics with the Calibrachoa scion. In some embodiments, the rootstock belongs to the Solanaceae family and more particularly, to the Petunia genus.

The term “scion” as used hereinafter refers to the plant part grafted onto the rootstock. The scion usually comprises the stem part or aerial parts (branches, leaves, flowers, fruits) of another plant. The scion is selected for it's properties such as the photosynthetic activity and the fruit or decorative properties.

The term “normal development” as used hereinafter refers to the growth parameters of the grafted scion. It is within the scope of the present invention that the growth and development of the grafted Calibrachoa scion is evaluated by comparing it the growth and development of a corresponding Calibrachoa plant (same species or same propagation material source) which is not subjected to a grafting procedure, namely, a non-grafted corresponding Calibrachoa plant. When the grafting process is successful and achieves sufficient compatibility between the scion and rootstock, the development of the scion, for example in terms of flower density and absence of chlorosis, is similar to the development of a corresponding non-grafted plant; in some cases the development of the grafted plant is enhanced relative to a corresponding non-grafted plant.

The term “source plant” or “mother plant” is herein defined as the initial grafting of a scion onto the rootstock in order to produce the “crop” of cuttings. Typically the source plant therefore should provide a large amount of vigorous cuttings which are subsequently used for future grafting or grown directly on their own root system. It is further within the scope of the present invention that the terms ‘stock plant’ or ‘source plant’ or ‘mother plant’ refer to the general process of vegetative propagation that enable to substantially maintain the specific genetic content of the desired variety and avoid changes in genotypes during reproductive stage. In some aspects it refers to the use of the grafting technology to create improved mother plants.

The term “compatibility” as used hereinafter, is defined as a sufficiently close genetic (taxonomic) relationship between stock and scion for a successful graft union or a uniform adhesion point to form, assuming that all other factors and parameters (technique, temperature, etc.) are satisfactory. It is herein further submitted that the degree of “relatedness” necessary for compatibility may vary with different taxa (species, genera, and families).

According to some aspects of the present invention, compatible characteristics between the rootstock and scion include, but are not limited to at least one characteristic selected from the group consisting of: similar stem or shoot diameter, similar growth pattern, taxonomical similarity, genetic similarity, anatomical similarity, formation of uniform adhesion point between said rootstock and said scion, absence of noticeable callus, suppressed formation of side shoots from the stem, suppressed formation of sprouts from the rooting callus, a thick and woody stem, normal development of said scion similar to a corresponding none grafted control plant, and any combination thereof. It is noted that the development of sprouts from the scion at the grafting point is a desirable characteristic, when normal branches are developed.

It is further within the scope of the invention, that the term “incompatibility” may herein be defined as failure (immediate or delayed) of a graft union to form. Incompatibility or failure of the graft to form may occur due to insufficiently close genetic relationship between the stock and scion, due to the grafting technique, cellular physiological intolerance that may be caused by metabolic, developmental and/or anatomical differences between stock and scion, failure to form functional vascular connections between stock and scion non-optimal environment, or disease such as viral disease, introduced as a result of grafting.

It is further within the scope that the term “immediate incompatibility” is herein defined as the rapid death of the scion characterized anatomically by lack or incomplete progression of graft union formation. Early events in normal graft union formation may include callus formation and formation of fibers.

The term “delayed incompatibility” refers to survival or normal growth of the grafted scion for months or even years, including normal formation of xylem, phloem and periderm, before degeneration of the graft union occurs.

The term “low to moderate incompatibility” refers hereinafter to a characteristic of a grafted plant in which the scion continues to develop after grafting with the rootstock. The scion development may be at a lower rate, or may expresses minimal morphological changes, relative to a non-grafted corresponding plant.

In general, the compatibility between the scion and the selected rootstock may be affected by the plant species, type of graft and environmental conditions before and/or following grafting. Such conditions may include temperature, which effects callus production, moisture, which effects cell enlargement in the callus bridge, growth pattern of the rootstock and virus contamination, insects and diseases.

It is herein acknowledged that the formation of a graft union requires the development of a de novo formed meristematic area between the scion (i.e. Calibrachoa scion) and the rootstock. According to some aspects, this process may comprise steps of: adhesion of the rootstock and the scion; proliferation of the callus at the interface and forming a callus bridge; and vascular differentiation across the graft interface.

The term “output” is herein generally defined as the percent of well-developed plants, rootstocks and scions, from the total grafted plants, under normal growth conditions.

The term “horticultural characteristics” as used herein generally refers to aspects of agriculture concerned with garden plant growing, and in the context of the present invention, especially ornamental and flower plants. It includes the production, cultivation, improvement, conservation, ornamental design, construction, maintenance and arboriculture of plants, especially ornamentals. It further includes all products, raw or processed, that arise from the horticultural industry.

It further involves plant propagation and cultivation with the aim of improving plant growth, yields, quality, ornamental design, and resistance to insects, diseases, and environmental stresses.

The term “conditioning” is herein generally refers to the various treatments that have been attempted or developed or designed and applied to the grown rootstock or scion or grafted plant or propagation material thereof, to improve compatibility between the rootstock and scion and growth and development of the grafted plant.

The term “propagation material” or “grafted propagation material” is herein refers to any raw material that is used for the creation of new plants by grafting. The propagation material or raw material encompasses seeds, all plant material or vegetative parts of plants intended for the production of the grafted plants. It is within the context of the present invention that such propagation material includes (1) rooted grafted propagation material e.g. young plants, and (2) unrooted grafted raw material or propagation material e.g. unrooted cuttings. The rooted and unrooted propagation material is used by growers to produce and develop the finished grafted Calibrachoa product.

The term “young plant” is herein defined as rooted propagation material comprising members of the following: rooted cuttings, seedlings, grafted rooted cuttings, and grafted seedlings. In a specific embodiment, a young plant refers to a small, young age plant with an active root system. The young plants as defined above can be planted and grown in any container, such as planting containers, pots, planters or flower beds. According to certain aspects, it is considered as an advanced level grafted propagation material, or, in other words it is considered as the most developed grafted raw material that finishers use, i.e. pot plant producers, growers that plant them in the field or in commercial planters or plantation, who grow them till harvesting stage. Young plant producers might graft the young plants whilst in the plug trays.

The term “unrooted propagation material” or “unrooted raw material” is herein defined as comprising members of the following: unrooted cuttings and unrooted grafted cuttings. It is within the scope of the present invention that such unrooted propagation material can be planted and grown in any growing container, but mostly grown in plug trays for the development of roots and thereafter may be transplanted by finishers into pots or planting containers for further growth and optionally thereafter delivered to retailers to be sold on to the consumers.

The present invention provides novel Calibrachoa grafted propagation material in the form of young plants or unrooted grafted raw material that is transported to its final position within a growing container or plantation to achieve a finished plant.

The term “finished plant” is herein defined as a grafted plant in its final size and design suitable for marketing to the late stage in the supply chain such as end user or florists.

The term “high stem” used herein, is to be understood in its common meaning in horticulture, namely an elongated stem of a plant bearing a “crown” on its top end. The crown of a plant refers to the plant's aboveground parts, including stems, leaves, and reproductive structures.

The term “high stem grafted Calibrachoa” used hereinafter refers to a rootstock grown in predetermined growth conditions designed to produce stem height of up to 50 cm or more, particularly between about 10 cm and 30 cm, upon which a Calibrachoa scion is engrafted. The resultant grafted Calibrachoa plant is advantageous by its enhanced stem height, vigorous growth and strong root system as compared to ungrafted normal Calibrachoa plant, which has a relatively weak root system and is susceptible to alkaline surroundings. The high stem grafted Calibrachoa can be produced from rooted or unrooted cuttings or from seedlings, and includes within it's scope high stem grafted propagation material or raw material.

It is further within the scope that the present invention provides novel Calibrachoa high stem grafted propagation material or raw material. Such high stem grafted propagation material comprises (1) high stem grafted young plants e.g. in the form of rooted cuttings, seedlings, grafted rooted cuttings, and grafted seedlings and (2) high stem grafted unrooted raw material e.g. in the form of unrooted cuttings and unrooted grafted cuttings.

It is according to a further embodiment that new propagation material comprising high stem grafted young plants and high stem grafted unrooted raw material is produced by the grafting method of the present invention and is an embodiment of the present invention.

In certain aspects, the present invention provides propagation material products, such as high stem grafted young plants and high stem grafted unrooted raw material in plug trays.

According to a further aspect, the present invention provides plugs of grafted Calibrachoa unrooted propagation material or raw material. The term “plug” or “plug tray” generally refers in horticulture to small-sized seedlings or cuttings grown in trays, filled usually with a peat or compost substrate. This type of plug may generally be used for commercially raising vegetables, ornamental plants and bedding plants. Plug plants are unrooted young plants raised in small, individual cells, for the development of roots, ready to be transplanted into containers, pots or a garden or fields.

It is within the context of the present invention to provide grafted Calibrachoa propagation material in the form of unrooted raw material and high-stem grafted propagation material in the form of unrooted raw material that is adapted to be inserted or rooted in plug trays. The unrooted propagation material is adapted to be sturdily, perpendicularly and stably grown in the plug trays until transplanted (e.g. by finishers) into pots or growing containers or any final commercial planter.

The term “sturdily grown” or “sturdiness” as used in the context of the present invention refers to the provision of a strong, tall stem or trunk for certain ornamental shrubs and trees. Generally in these cases, a graft is made at a desired height on a stock plant, with a strong stem. This is used to raise plants, usually ornamental plants with a shrubby or bushy growth habit or ornamental trees, on a high stem to produce a new combination

The term “cutting” or “cuttings” as used herein refers to vegetative or asexual propagating plant material. Plant cuttings are used for vegetative (asexually) propagating plants by a technique in which a piece of the stem, leaves or root of the source plant is placed in a suitable medium such as moist soil, potting mix, coir or rockwool. The cutting produces new roots, stems, or both, and thus becomes a new plant independent of the parent or source plant. Plant cutting technique is also known as striking or cloning technique. In the context of the present invention, the term “cutting” or “cuttings” include rooted cuttings, unrooted cuttings, grafted rooted cuttings and grafted unrooted cuttings.

The term “unrooted cutting” refers to cuttings that have no roots and must be inserted in the plug tray medium (the soil) and take root there.

The term “seedlings” as used herein refers to a young plant developing from a seed and includes seedlings and grafted seedlings.

In the context of the present invention, the term “variety” or “varieties” correspond to the usual denomination in agricultural industry and correspond to a plant of a given botanical taxon which is distinct from other existing plant, which is uniform and stable.

The term “normal Calibrachoa plant” used hereinafter refers to an ungrafted or non-grafted plant of the Calibrachoa genus in its natural growth form. This term also encompass the terms ‘normal plant’ and ‘a corresponding non-grafted plant’ or ‘a corresponding non-grafted control plant’.

The term “etiolated plant” or “etiolating plants” used hereinafter refers to flowering plants grown in partial or complete absence of light. These plants are generally characterized by elongated stems; longer internodes, hence usually fewer leaves per unit length of stem and a pale yellow color (chlorosis). According to one embodiment, the selected rootstock plants of the present invention are grown under low radiation conditions that enhance the production of etiolated plants.

According to one embodiment, the present invention surprisingly provides for the first time a grafted Calibrachoa plant. The grafted plant comprises a Calibrachoa scion engrafted upon any selected rootstock. In preferred embodiments, the selected rootstock is compatible with the Calibrachoa scion.

According to a further embodiment the grafted plant according to the present invention is obtained by grafting a Calibrachoa scion with any compatible rootstock, provided they are compatible in term of grafting-ability, and that the selected rootstock provides predetermined desirable characteristics such as strong root system, vigorous growth, tolerance to at least one biotic and/or abiotic stress and/or enhanced stem height.

The Calibrachoa grafted plant of the present invention may be derived from propagation material or raw material comprising members of the following: young plants, namely rooted cuttings, seedlings, grafted rooted cuttings, and grafted seedlings, or from unrooted propagation material or raw material comprising members of the following: unrooted cuttings and grafted unrooted cuttings. Thus the present invention provides young plants or any other propagation material or raw material comprising rooted cuttings, unrooted cuttings, seedlings, grafted rooted cuttings, grafted unrooted cuttings and grafted seedlings useful for producing grafted Calibrachoa plants, including high stem grafted Calibrachoa.

In certain embodiments, the rootstock can be selected from the group comprising Solanum, Capsicum, Petunia and Calibrachoa genus.

The present invention further encompasses plants comprising a rootstock engrafted with at least two different scions belonging to two different species or varieties.

It is to be understood that the invention is not limited to the examples described herein, but it should be considered with any sorts of compatible rootstocks, and for any sorts of grafting methods. Thus, the invention is capable of other embodiments carried out in various ways.

In one embodiment, the present invention discloses means and methods for grafting between genera within the same family.

According to a further embodiment, the present invention discloses means and methods for grafting between species within a genus.

In a more preferred embodiment, the selected rootstock to be grafted with the Calibrachoa scion is a variety or cultivar of the Solanaceae family.

Accordingly, the engrafted plant according to the present invention can comprise a Solanaceae rootstock variety or cultivar. These rootstock varieties or cultivars may be any type of the Solanaceae family, provided they are compatible for grafting with the Calibrachoa scion.

In a further embodiment, the plant according to the present invention comprises at least two scions engrafted on a compatible rootstock.

In a further embodiment, the present invention provides a grafted Calibrachoa plant. The aforementioned plant comprises a rootstock engrafted with a Calibrachoa scion.

It is further within the scope that the rootstock is compatible with the Calibrachoa scion.

It is according to another embodiment of the invention, wherein said plant is derived from propagation material in the form of young plants or any other raw material selected from the group consisting of rooted cuttings, unrooted cuttings, seedlings, grafted rooted cuttings, grafted unrooted cuttings and grafted seedlings.

In accordance with a certain aspect of the present invention the plant as defined in any of the above is derived from propagation material selected from the group consisting of young plants and unrooted raw material.

In accordance with a further aspect of the present invention such young plants are selected from the group consisting of rooted cuttings, seedlings, grafted rooted cuttings, and grafted seedlings.

In accordance with a further aspect of the present invention the unrooted raw material is selected from the group consisting of unrooted cuttings and unrooted grafted cuttings.

In accordance with a further aspect of the present invention the unrooted raw material is adapted for insertion and growth in plug trays.

Another embodiment of the present invention is the provision of a finished plant derived from the propagation material or young plant or raw material as defined above.

It is according to another embodiment of the invention, wherein said rootstock is derived from propagation material selected from the group consisting of seeds, rooted cuttings, unrooted cuttings, seedlings, grafted rooted cuttings, grafted unrooted cuttings and grafted seedlings.

It is according to another embodiment of the invention, wherein the rootstock belongs to the Solanaceae family.

It is according to another embodiment of the invention, wherein the rootstock is selected from the group consisting of Cestroideae, Goetzeoideae, Nicotianoideae, Petunioideae, Schizanthoideae, Schwenckioideae and Solanoideae subfamily.

It is according to another embodiment of the invention, wherein the rootstock is selected from the group consisting of Solanum, Capsicum, Petunia and Calibrachoa genus.

It is according to another embodiment of the invention, wherein the rootstock is selected from the group consisting of Petunia alpicola, P. altiplana, P. axillaris, P. bajeensis, P. bonjardinensis, P. exserta, P. guarapuavensis, P. helianthemoides, P. humifusa, P. inflate, P. integrifolia, P. interior, P. ledifolia, P. littoralis, P. mantiqueirensis, P. occidentalis, P. parviflora, P. patagonica, P. pubescens, P. reitzii, P. riograndensis, P. saxicola, P. scheideana, P. variabilis, P. villadiana, P.×atkinsiana and P. hybrida species.

It is still within the scope, that rootstock is selected from the group consisting of Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia, Calibrachoa×hybrida and Petchoa species.

It is still within the scope, that rootstock is selected from the group consisting of lycopersicum, tuberosum and melongena species.

It is according to another embodiment of the invention, wherein the scion is derived from seeds, rooted cuttings, unrooted cuttings, seedlings, grafted rooted cuttings, grafted unrooted cuttings and grafted seedlings.

It is according to another embodiment of the invention, wherein the scion is of the Calibrachoa genus.

It is further within the scope that scion is selected from the group consisting of Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia species.

It is according to another embodiment of the invention, wherein the rootstock has at least one compatible characteristic with the scion.

It is further within the scope that a unique growth protocol has been designed and performed for each rootstock genus or species grown until the grafting stage. The growth protocol includes parameters such as predetermined irrigation intervals, predetermined illuminating and shading levels, temperature, pH, relative humidity, electrical conductivity and predetermined fertilization levels.

It is according to another embodiment of the invention, wherein the at least one compatible characteristic is selected from the group consisting of: hypocotyl length, stem diameter, nodes number, internode length, growth pattern, taxonomical similarity, genetic similarity, anatomical similarity and any combination thereof.

It is according to another embodiment of the invention, wherein the rootstock has desirable agronomical characteristics.

It is according to another embodiment of the invention, wherein the desirable agronomical characteristics are selected from the group consisting of strong root system, enhanced stem height, plant vigor, stress tolerance, disease resistance, resistance to pathogens, resistance to insect infestation, resistance to abiotic stresses and any combination thereof.

It is according to another embodiment of the invention, wherein the engrafted Calibrachoa plant has desirable ornamental and/or agronomical characteristics relative to a normal ungrafted Calibrachoa plant selected from the group consisting of: enhanced height or high stem plant, strong root system, tolerance to biotic stress, tolerance to abiotic stress, uniformity, reduced leaf yellowing, robust growth, enhanced inflorescence yield, enhanced yield of plant propagation material, enhanced aerial yield, combining more than one Calibrachoa species or variety on one plant and any combination thereof.

It is according to another embodiment of the invention, wherein the stress tolerance comprises an abiotic stress tolerance selected from the group consisting of cold tolerance, high temperature tolerance, drought tolerance, salt tolerance, pH tolerance and any combination thereof.

It is according to another embodiment of the invention, wherein the stress tolerance comprises a biotic stress tolerance selected from the group consisting of a disease resistance, an insect resistance, and a nematode resistance, improved resistance to soil borne pathogens and any combination thereof.

It is according to another embodiment of the invention, wherein said scion is engrafted at a height of up to 50 cm on said rootstock.

It is according to another embodiment of the invention, wherein said rootstock is engrafted with at least two different scions said scions belong to different varieties and are compatible with said rootstock.

It is according to another embodiment of the invention, wherein said different scions have at least one different characteristic selected from the group consisting of: inflorescence color, fruit shape, growth pattern and any combination thereof.

It is according to another embodiment of the invention, wherein said engrafted Calibrachoa plant has desirable ornamental and/or agronomical characteristics relative to ungrafted Calibrachoa plant selected from the group consisting of: enhanced height or high stem plant, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof.

It is according to another embodiment of the invention, wherein said plant is planted into a final commercial planter or pot or any other growing container.

It is according to another embodiment of the invention, wherein said final commercial planter or pot or any other growing container has a pot size of 1 liter and less.

It is according to another embodiment of the invention, wherein said final commercial planter or pot or any other growing container has a pot size of 1 liter and more.

It is according to another embodiment of the invention, wherein said plant is a high stem grafted Calibrachoa adapted for perpendicular stability and sturdiness when planted in a pot.

It is according to another embodiment of the invention, wherein said plant is derived from propagation material or young plants or raw material selected from the group consisting of rooted cuttings, unrooted cuttings, seedlings, grafted rooted cuttings, grafted unrooted cuttings and grafted seedlings.

It is according to another embodiment of the invention, wherein said young plant is adapted for perpendicular stability and/or sturdiness when planted in a plug tray.

It is according to another embodiment of the invention, wherein said plant is derived from propagation material selected from the group consisting of high stem grafted young plants and unrooted high stem grafted raw material.

It is according to another embodiment of the invention, wherein said high stem young plants are selected from the group consisting of rooted cuttings, seedlings, grafted rooted cuttings, and grafted seedlings.

It is according to another embodiment of the invention, wherein said high stem unrooted raw material is selected from the group consisting of unrooted cuttings and unrooted grafted cuttings.

It is according to another embodiment of the invention, wherein said high stem unrooted raw material is adapted for perpendicular stability and/or sturdiness when planted in a plug tray.

It is according to another embodiment of the invention to provide a finished plant derived from the propagation material as defined in any of the above.

It is according to another embodiment of the invention, wherein the finished plant is adapted for perpendicular stability and/or sturdiness when planted in a pot or planting container.

It is according to another embodiment of the invention to provide cuttings or unrooted cuttings, grafted cuttings or grafted unrooted cuttings of the high stem grafted Calibrachoa as defined in any of the above.

It is according to another embodiment of the invention to provide any part of the plant as defined in any of the above.

It is according to another embodiment of the invention, wherein said plant part is selected from the group consisting of grafted cuttings, unrooted grafted cuttings, flowers and any other vegetative or reproductive part of the plant.

According to a further aspect of the invention, the production of grafted Calibrachoa plants or young plants enables the provision of a new marketable product comprising an enlarged and varied scope of commercially valuable Calibrachoa plant designs and products. The grafted Calibrachoa plants of the present invention have significant added values with respect to unique, novel and commercially desirable ornamental designs and combinations, which surprisingly overcome inherent undesirable characteristics of plants of the Calibrachoa genus (e.g. crawling growth pattern, weak root system, poor plant vigor, sensitivity to heat, alkalinity and salt etc.).

According to a further aspect, the grafted Calibrachoa plants of the present invention are advantageous with respect to their yield, productivity and cost benefit ratio. By grafting Calibrachoa on predetermined rootstock, the provision of Calibrachoa plants with enhanced plant vigor is achieved, such that significantly higher yield of vegetative cuttings can be produced from the grafted plant, relative to the Calibrachoa plant from which it was derived. Thus the grafted plant can supply high yield of propagated plant material which grows into an exact copy of the desirable parental plant. Therefore the efficiency in production of useful and desirable Calibrachoa cuttings, propagated material and young plants is enhanced and the production costs are reduced by grafting Calibrachoa upon a selected rootstock with improved plant vigor characteristics.

According to a further aspect, the present invention provides grafted propagation material of the plant as defined in any of the above, wherein said propagation material is selected from the group consisting of grafted young plants, grafted rooted cuttings, grafted unrooted cuttings, grafted seedlings, high stem grafted young plants and high stem grafted unrooted cuttings.

The present invention further provides a method for producing a Calibrachoa engrafted plant. The aforementioned method comprises steps of: (a) providing a scion of the genus Calibrachoa; (b) selecting a rootstock having compatibility with said Calibrachoa scion; (c) growing said rootstock under agrotechnical conditions so as to have growth characteristics suitable for grafting onto said scion; and (d) grafting said scion onto said rootstock so as to produce a grafted Calibrachoa plant.

It is further within the scope to provide the method as described above, further comprising steps of growing propagation material or young plants or raw material selected from the group consisting of rooted cuttings, unrooted cuttings, seedlings, grafted rooted cuttings, grafted unrooted cuttings and grafted seedlings at predetermined growth conditions so as to provide said Calibrachoa grafted plant.

It is further within the scope to provide the method as described above, further comprising steps of producing and growing propagation material selected from the group consisting of young plants and unrooted raw material at predetermined growth conditions so as to provide said Calibrachoa grafted plant.

It is further within the scope to provide the method as described above, further comprising steps of selecting said young plants from the group consisting of rooted cuttings, seedlings, grafted rooted cuttings, and grafted seedlings.

It is further within the scope to provide the method as described above, further comprising steps of selecting said unrooted raw material from the group consisting of unrooted cuttings and unrooted grafted cuttings.

It is further within the scope to provide the method as described above, further comprising steps of growing said unrooted raw material in plug trays.

It is further within the scope to provide the method as described above, further comprising steps of growing said propagation material or young plants or raw material so as to produce a finished plant.

It is further within the scope to provide the method as described above, further comprising steps of growing propagation material or young plants or raw material selected from the group consisting of rooted cuttings, unrooted cuttings, seedlings, grafted rooted cuttings, grafted unrooted cuttings and grafted seedlings of said rootstock at predetermined growth conditions so as to be suitable for grafting with said scion.

It is further within the scope to provide the method as described above, further comprising steps of growing propagation material or young plants or raw material selected from the group consisting of rooted cuttings, unrooted cuttings, seedlings, grafted rooted cuttings, grafted unrooted cuttings and grafted seedlings of said scion at predetermined growth conditions so as to be suitable for grafting with said rootstock.

It is further within the scope to provide the method as described above, comprising additional steps of adjusting the density of the grown rootstock plants by controlling the number of plants per area.

It is further within the scope to provide the method as described above, comprising additional steps of selecting a suitable growth tray by parameters selected from the group consisting of size, shape of cells, material, density and any combination thereof.

It is further within the scope to provide the method as described above comprising additional steps of applying controlled Low-Radiation regimes for obtaining etiolated rootstock plants.

It is further within the scope to provide the method as described above, comprising additional steps of controlling the flowering timing of the grown rootstock plants by affecting photoperiodic response of the plants.

It is further within the scope to provide the method as described above, comprising additional steps of using trailing and stabilizing means for supporting and shaping the grown rootstock.

It is further within the scope to provide the method as described above, comprising additional steps of using trailing and stabilizing means selected from the group consisting of sticks, clips, flowers cutting, trailing net, plastic tray frame and any combination thereof.

It is further within the scope to provide the method as described above, comprising additional steps of growing the selected rootstock plants at a predetermined protocol by controlling parameters selected from the group consisting of temperature, relative humidity, electrical conductivity, illuminating level, shading level, pH, irrigation intervals, fertilization solutions and regimes and any combination thereof.

It is further within the scope to provide the method as described above, comprising additional steps of applying plant protection treatments selected from the group consisting of: pesticide-based treatment, herbicides treatment, insecticides treatment, fungicides treatment, biological pest control treatment and any combination thereof.

It is further within the scope to provide the method as described above, comprising additional steps of coordinating at least one property of the plant propagation material to be grown for rootstock production, said at least one property is selected from the group consisting of root system development, hypocotyl size, hypocotyl length, stem diameter, nodes number, internode length and any combination thereof.

It is further within the scope that the rootstock plant propagation material of the present invention is conditioned and selected for root system development properties such as development rate, conditions for development, root system size, functionality and rooting output.

It is further within the scope to provide the method as described above, further comprises steps of enhancing adhesion between said scion and said rootstock to form a graft union by regulating at least one agrotechnical or environmental parameter selected from the group consisting of: temperature, humidity, moisture, pH, illuminating, shading and any combination thereof.

It is further within the scope to provide the method as described in any of the above, comprising additional steps of growing rooted cuttings or unrooted cuttings of said Calibrachoa scion at predetermined growth conditions so as to provide said Calibrachoa scion suitable for grafting onto said rootstock.

It is further within the scope to provide the method as described in any of the above comprising additional steps of growing seeds, seedlings, rooted cuttings or unrooted cuttings of said rootstock at predetermined growth conditions so as to be suitable for grafting with said scion.

It is further within the scope to provide the method as described in any of the above comprising additional steps of growing said rootstock to a predetermined height of up to 50 cm.

It is further within the scope to provide the method as described in any of the above comprising additional steps of growing said rootstock at predetermined growth conditions selected from the group consisting of: irrigation intervals, fertilization solutions and regimes, illuminating and shading regimes and any combination thereof.

It is further within the scope to provide the method as described in any of the above comprising additional steps of manipulating the growth of said rootstock plant so as to have growth characteristics suitable for grafting with said scion, said growth characteristics are selected from the group consisting of: stem diameter, stem height, growth pattern and any combination thereof.

It is further within the scope to provide the method as described in any of the above further comprising steps of manipulating and coordinating the growth of said rootstock plant so as to achieve growth characteristics suitable for grafting with said scion, said growth characteristics are selected from the group consisting of: hypocotyl size, hypocotyl length, stem diameter, nodes number, node length, stem diameter, stem height, growth pattern, flowering time and any combination thereof.

It is further within the scope to provide the method as described in any of the above comprising additional steps of grafting said scion onto said rootstock by a grafting technique selected from the group consisting of: splice grafting, bud grafting, cleft grafting, side grafting, approach grafting, hole insertion grafting, one cotyledon grafting, whip grafting, stub grafting, four flap grafting, awl grafting, veneer grafting and by any other grafting technique suitable for grafting herbaceous plant and any combination thereof.

It is further within the scope to provide the method as described in any of the above comprising additional steps of grafting said scion onto said rootstock by cleft grafting.

It is further within the scope to provide the method as described in any of the above comprising additional steps of planting said grafted plant into a final commercial planter or pot or any other growing container.

It is further within the scope to provide the method as described in any of the above comprising additional steps of planting said grafted plant into a final commercial planter or pot or any other growing container having a pot size of 1 liter and less.

It is further within the scope to provide the method as described in any of the above comprising additional steps of planting said grafted plant into a final commercial planter or pot or any other growing container having a pot size of 1 liter and more.

It is further within the scope to provide the method as described in any of the above, further comprises a step of producing an engrafted Calibrachoa plant with desirable ornamental and/or agronomical characteristics relative to ungrafted Calibrachoa plant selected from the group consisting of: enhanced height or high stem plant, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof.

It is further within the scope to provide the method as described in any of the above, further comprising steps of producing a high stem grafted Calibrachoa.

It is further within the scope to provide the method as described in any of the above, further comprising steps of growing the rootstock to a predetermined height of at least about 10 cm and predetermined stem diameter and grafting the Calibrachoa scion upon said rootstock at said predetermined height.

It is further within the scope to provide the method as described in any of the above, further comprising steps of producing and growing propagation material or young plants or raw material selected from the group consisting of rooted cuttings, unrooted cuttings, seedlings, grafted rooted cuttings, grafted unrooted cuttings and grafted seedlings at predetermined growth conditions so as to provide said high stem grafted Calibrachoa plant.

It is further within the scope to provide the method as described in any of the above, further comprising steps of planting said high stem grafted Calibrachoa young plant in a plug tray.

It is further within the scope to provide the method as described in any of the above, wherein the unrooted propagation material or raw material is adapted for perpendicular stability and/or sturdiness when planted in a plug tray.

It is further within the scope to provide the method as described in any of the above, further comprising steps of producing and growing propagation material selected from the group consisting of high stem young plants and unrooted high stem raw material at predetermined growth conditions so as to provide said Calibrachoa high stem grafted plant.

It is further within the scope to provide the method as described in any of the above, further comprising steps of selecting said high stem young plants from the group consisting of rooted high stem cuttings, high stem seedlings, grafted high stem rooted cuttings, and grafted high stem seedlings.

It is further within the scope to provide the method as described in any of the above, further comprising steps of selecting said high stem unrooted raw material from the group consisting of unrooted high stem cuttings and unrooted high stem grafted cuttings.

It is further within the scope to provide the method as described in any of the above, further comprising steps of planting said high stem unrooted high stem raw material in plug trays.

It is further within the scope to provide the method as described in any of the above, further comprising steps of growing said high stem unrooted high stem raw material with perpendicular stability and/or sturdiness in plug trays.

It is further within the scope to provide the method as described in any of the above, further comprising steps of growing said propagation material so as to produce a finished high stem grafted Calibrachoa plant.

It is further within the scope to provide any part of a plant produced by the method as defined in any of the above.

It is further within the scope to provide the method as described in any of the above, wherein said plant part is selected from the group consisting of rooted cuttings, unrooted cuttings, grafted rooted cuttings and grafted unrooted cuttings, flowers and any other vegetative or reproductive part of the plant as defined in any of the above.

It is further within the scope to provide grafted propagation material of a plant produced by the method as defined in any of the above, wherein said propagation material is selected from the group consisting of grafted young plants, grafted rooted cuttings, grafted unrooted cuttings, grafted seedlings, high stem grafted young plants and high stem grafted unrooted cuttings.

It is further within the scope to provide the method as described in any of the above comprising additional steps of engrafting said rootstock with at least two different scions.

It is further within the scope to provide the method as described in any of the above comprising additional steps of engrafting said rootstock with at least two different scions, said scions belong to different varieties and are compatible with said rootstock.

It is further within the scope to provide the method as described in any of the above comprising additional steps of providing said different scions having at least one different characteristic selected from the group consisting of: inflorescence color, fruit shape, growth pattern and any combination thereof.

In order to understand the invention and to see how it may be implemented in practice, a plurality of preferred embodiments will now be described, by way of non-limiting example only, with reference to the following examples.

### Example 1

### A Process for Producing High-Stem Grafted Calibrachoa Plant

1. Screening for sources of plant propagation material:

The first step was identifying source material, including different species of seeds and/or cuttings propagation material.

A preliminary screening of different genus of the family Solanaceae identified species of the Calibrachoa and Petunia genus that have been selected as genetic source material for the rootstock and scion species.

From the Calibrachoa and Petunia genus, 6 varieties have been selected for further tests, according to their horticultural traits and evaluation of their potential suitability for the desired grafted plant end product (see Table 1).

TABLE 1
Description of examples of propagation material

| | Commercial | | | Main desired | | |
|---|---|---|---|---|---|---|
| Variety | name | model | Use | qualities | uniformity | availability |
| Petunia surfinia | Surfinia | White; | Rootstock | Long internodes; | High | High |
| | | Burgundy; Hot | | Robustness | | |
| | | pink; Purple; | | | | |
| | | Dark violet; | | | | |
| | | Yellow; Blue | | | | |
| | | spritz; Pink | | | | |
| | | spritz | | | | |
| Petunia sp. | Happy Giant | Hot pink | Rootstock | Long internodes; | High | High |
| | | | | Robustness | | |
| Petunia sp. | Bingo | White; Blue; | Rootstock | Long internodes; | Low | High |
| | | Purple | | Robustness | | |
| Petunia sp. | Crazytunia | Mandeville; | Rootstock | Robustness | Low | Low |
| | | Stone-wash | | | | |
| Calibrachoa sp. | Chameleon | Lavender; | Scion | Dense flowering; | High | Low |
| | | Purple; Pink | | Unique colors | | |
| | | Passion | | | | |
| Calibrachoa sp. | Celebration ® | White improved; | Scion | Dense flowering; | High | High |
| | | Yellow banana; | | Unique colors | | |
| | | Neon rose; | | | | |
| | | Mandarin; | | | | |
| | | Cherry; Violet | | | | |

In addition, a screening of 70,000 breeding products and selection of candidates to be used as source material for rootstock and scion has been performed. Four rootstock candidates have been selected for further tests as described in Table 2.

Table 2
Selected varieties of Petunia, to be tested as a rootstock

| | | | Rootstock candidate: Main | | |
|---|---|---|---|---|---|
| | | | desired qualities | | |
| | Experimental | | Long | | Sparse |
| Variety | name* | model | Internodes | thickness | Flowering |
| Petunia | 14-1914 | Pink | + | +++ | + |
| sp. | | | | | |
| Petunia | 14-1915 | Red star | ++ | ++ | ++ |
| sp. | | | | | |
| Petunia | 14-1916 | Purple-pink | +++ | + | +++ |
| sp. | | | | | |
| Petunia | 14-1917 | Purple | +++ | ++ | +++ |
| sp. | | | | | |

*Experimenal name by Westhoff Vertriebsges. mbH

2. Screening for candidate species to be used for scion and rootstock grafting.

Morphological identification of 10 Petunia species engrafted with Calibrachoa sp. i.e. chameleon has been performed according to parameters such as: rooting potential, rooting quality, callus formation, sprouts from the callus and side-shoots development from the stem, as presented in Table 3. The test has been conducted twice, 10 replicates per rootstock.

The desirable qualities that the different rootstocks have been screened for included: maximal rooting potential, minimal sprout differentiation from callus and/or minimal side-shoots from the stem, and maximal stem thickness and elongation of internodes.

TABLE 3
Effect of grafting Calibrachoa sp. on different Petunia rootstocks, on
rootstock quality parameters

| | | | | | | | Average |
|---|---|---|---|---|---|---|---|
| | | | | | | Average | elongation |
| | | | Rooting | Sprout | Side-shoots | stem | of |
| | Commercial | | potential | differentiation from | from the | thickness | internodes |
| Var | name | model | (%) | callus (%) | stem (%) | (mm) | (mm) |
| Petunia surfinia | Surfinia | White | 95 | 100 | 80 | 5 | 25 |
| Petunia surfinia | Surfinia | Burgundy | 95 | 80 | 80 | 8 | 10 |
| Petunia surfinia | Surfinia | Hot pink | 95 | 50 | 80 | 8 | 30 |
| Petunia surfinia | Surfinia | Purple | 95 | 10 | 80 | 7 | 10 |
| Petunia surfinia | Surfinia | Dark violet | 95 | 0 | 80 | 7 | 30 |
| Petunia surfinia | Surfinia | Yellow | 95 | 90 | 80 | 6 | 10 |
| Petunia surfinia | Surfinia | Blue spritz | 95 | 80 | 80 | 8 | 40 |
| Petunia surfinia | Surfinia | Pink spritz | 95 | 80 | 80 | 8 | 40 |
| Petunia sp. | Happy Giant | Hot pink | 95 | 50 | 80 | 10 | 50 |
| Petunia sp. | Bingo | White | 95 | 50 | 80 | 6 | 30 |
| Petunia sp. | Bingo | Blue | 95 | 60 | 80 | 8 | 30 |
| Petunia sp. | Bingo | Purple | 95 | 60 | 80 | 8 | 30 |
| Petunia sp. | Crazytunia | Mandeville | 95 | 30 | 80 | 7 | 40 |
| Petunia sp. | Crazytunia | Stone-wash | 95 | 30 | 80 | 7 | 30 |

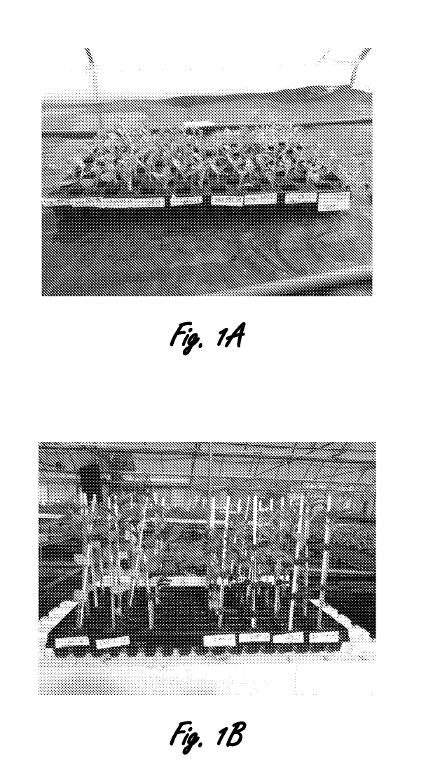

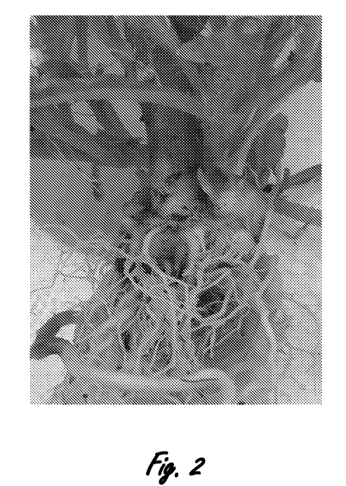

Reference is now made to FIG. 1 presenting a photographic illustration demonstrating a high rooting potential of different Petunia surfinia rootstocks (FIG. 1A) and a high stem Petunia rootstocks produced by means and methods inter alia described in the present invention (FIG. 1B). It is emphasized that the protocols disclosed in the present invention are adapted to achieve an elongated or stretched rootstock (as illustrated in FIG. 1B), with a high rooting potential (as illustrated in FIG. 1A) to be used for grafting with a desirable Calibrachoa scion. Such protocols include specific and predetermined fertigation and shading regimes to produce an elongated rootstock with minimal side shoots from the stem.

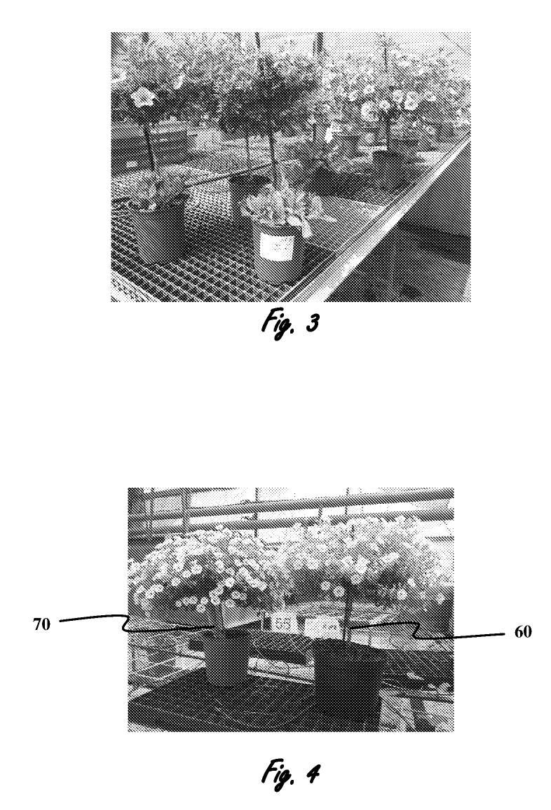

Reference is now made to FIG. 2 presenting a photographic illustration of callus formation, rooting and the undesirable phenomena of sprouts growth from the rooting callus of a Calibrachoa sp. grafted upon Petunia rootstocks.

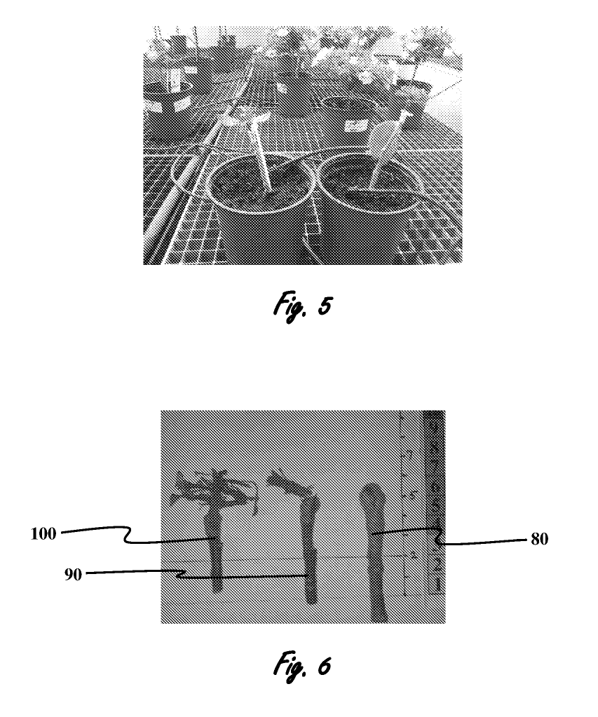

Reference is now made to FIG. 3 presenting a photographic illustration demonstrating growth of sprouts from callus of a Calibrachoa sp. grafted upon Petunia rootstocks.

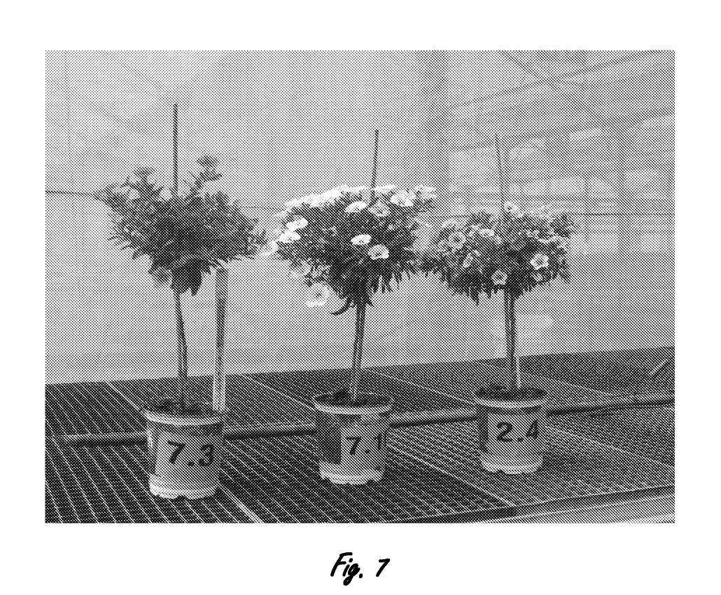

Reference is now made to FIG. 4 presenting a photographic illustration of trunk development and thickness, of different Petunia rootstocks engrafted with Calibrachoa sp. Celebration® yellow banana scion. A thin rootstock 60 (i.e. stem diameter of about 5 mm) is illustrated in a grafting product comprising Petunia surfinia Yellow as a rootstock, and a thick rootstock 70 (i.e. stem diameter of about 10 mm) is illustrated in a grafting product comprising Petunia Happy Giant Hot pink as a rootstock.

It is noted that it is an important aspect of the present invention to produce, select and screen for grafted Calibrachoa plants characterized by minimal sprout differentiation from the callus and/or minimal side-shoots from the stem, as shown in FIG. 2 and FIG. 3, respectively; and further characterized by maximal stem thickness and elongation of internodes as demonstrated in FIG. 4.

3. Evaluation of different scion and rootstock combinations for achieving a desirable horticultural product.

Parameters and results are presented in Table 4.

In Table 4: maximal rootstock thickness is defined as up to 10 mm in diameter, moderate rootstock development is defined as about 6 to 8 mm in diameter, well or good rootstock development is defined as a combination of characteristics comprising reduced side shoots, internodes longer than about 10 mm and diameter higher than about 5 mm, and moderate stem side-shoots and sprouts from callus is defined as between about 50% and 80% development of stem side-shoots and sprouts from callus.

Two approaches have been used for testing compatibility and grafting:

A. Grafting on short stem (4-7 cm) to evaluate compatibility between the rootstock and the scion and grafting potential output (output is herein generally defined as the percent of well-developed plants, rootstocks and scions, from total grafted plants, under normal growing conditions). Different levels for compatibility have been identified and their engrafted products were evaluated.

B. High stem grafting feasibility—grafting on long stem (about 15-18 cm) to evaluate physical and physiological parameters.

A further step performed is testing the life duration of different engrafted products by preserving the engrafted plants and growing them for up to additional 12 months.

TABLE 4
Grafting different Calibrachoa varieties on different Petunia rootstocks;
evaluation of engrafted products and rootstock-scion reciprocal effects

| | Rootstock | | Tested Scion | | |
|---|---|---|---|---|---|
| Rootstock | model | Tested Scion | models | Rootstock results | Scion results |
| Petunia surfinia | white | Calibrachoa sp. | Yellow banana; | Undeveloped, thin | Nutritional |
| | | Celebration ® | White improved; | (about 5 mm), sprouts | deficiency |
| | | | Neon rose; | from callus; Nutritional | (chlorosis); |
| | | | Mandarin; | deficiency (chlorosis) | normal physical |
| | | | | | development, |
| | | | Cherry; Violet | | similar to non- |
| | | | | | grafted control |
| Petunia surfinia | Burgundy; hot | Calibrachoa sp. | Pink; Purple | Moderate rootstock | Unaffected by |
| | pink; purple; | Chameleon | | development (between | rootstock; normal |
| | yellow; blue | | | 5 mm to 8 mm | development |
| | spritz; pink | | | thickness) sprouts from | similar to non- |
| | spritz | | | callus | grafted control |
| | Dark violet | Calibrachoa sp. | Pink; Purple | Moderate rootstock | Unaffected by |
| | | Chameleon | | development (between | rootstock; normal |
| | | | | 5 mm to 8 mm | development |
| | | | | thickness). No sprouts | similar to non- |
| | | | | from callus | grafted control |
| Petunia sp. Happy | Hot pink | Calibrachoa sp. | Pink; Purple | Good rootstock | Unaffected by |
| Giant | | Chameleon | | development (maximal | rootstock; normal |
| | | | | thickness of about10 | development |
| | | | | mm); moderate stem | similar to non- |
| | | | | side-shoots and sprouts | grafted control |
| | | | | from callus (about 50- | |
| | | | | 80%) | |
| Petunia sp. Happy | Hot pink | Calibrachoa sp. | mandarin; cherry | Good rootstock | Unaffected by |
| Giant | | Celebration ® | | development (maximal | rootstock; normal |
| | | | | thickness); moderate | development |
| | | | | stem side-shoots and | similar to non- |
| | | | | sprouts from callus | grafted control |
| | | | | (about 50-80%) | |
| Petunia sp. Bingo | White | Calibrachoa sp. | Pink; Purple | Undeveloped, thin, | Nutritional |
| | | Chameleon | | sprouts from callus; | deficiency |
| | | | | Nutritional deficiency | (chlorosis); |
| | | | | (chlorosis) | normal physical |
| | | | | | development, |
| | | | | | similar to non- |
| | | | | | grafted control |
| Petunia sp. Bingo | blue; Purple | Calibrachoa sp. | Pink; Purple | Good rootstock | Unaffected by |
| | | Chameleon | | development (well | rootstock; normal |
| | | | | thickness of about 8 | development |
| | | | | mm); stem side-shoots | similar to non- |
| | | | | and sprouts from callus | grafted control |
| Petunia sp. | Mandeville | Calibrachoa sp. | Pink; Purple | undeveloped, thin, | Moderate |
| Crazytunia | | Chameleon | | moderate stem side- | incompatibility, |
| | | | | shoots and sprouts | 70% output |
| | | | | from callus | |
| Petunia sp. | Stone-wash | Calibrachoa sp. | Pink; Purple | undeveloped, thin, | Moderate |
| Crazytunia | | Chameleon | | moderate stem side- | incompatibility, |
| | | | | shoots and sprouts | 70% output |
| | | | | from callus | |

4. Products of the grafting process: two rootstocks and four scions have been identified and selected as examples suitable for commercial protocol usage.

The Petunia sp. ‘Happy giant’ model ‘Hot pink’ has been found to be suitable as a rootstock. Its advantageous traits include: producing straight trunk, woody, having less nodes and secondary shoot growth from the callus bridge. This variety has been tested for grafting with 4 selected Calibrachoa sp. scions, i.e. ‘Chameleon’ model Pink and Purple and Celebration® model mandarin and cherry.

Petunia surfinia model ‘Dark violet’ was also found to be well suited to the scope of the present invention. It was grafted with the two tested scions, Calibrachoa sp. ‘Chameleon’ model Pink or Purple. The grafting process resulted in a desirable rootstock which was developed into a straight and woody trunk without any sprouts from the callus. The scions also developed well, with green color and dense flowering.

Incompatibility was recorded between the rootstocks Petunia sp. ‘Crazytunia’ models Mandeville and Stone-was, and the scions Calibrachoa sp. ‘Chameleon’ models Pink and Purple. As illustrated in FIG. 5, these grafted plants did not develop well, and 30% of them wilted.

5. Commercial development and evaluation.

5.1 Developing prototypes adapted for specific end users or retailers, for example, comprising Calibrachoa sp. ‘chameleon’ series as scions.

5.2 Performing adaptations to the market demands, for example, rootstock height and grafted head diameter were changed to provide optimal fit to the growing pot.

5.3 Engrafted products were subjected to commercial experimenters, for example:

A. The Petunia sp. ‘Happy giant’ model ‘Hot pink’ grafted with Calibrachoa sp. scion ‘Chameleon’ model Pink;

B. The Petunia sp. ‘Happy giant’ model ‘Hot pink’ grafted with Calibrachoa sp. scion ‘Chameleon’ model Purple;

C. The Petunia sp. ‘Happy giant’ model ‘Hot pink’ grafted with Calibrachoa sp. scion Celebration® model mandarin;

D. The Petunia sp. ‘Happy giant’ model ‘Hot pink’ grafted with Calibrachoa sp. scion Celebration® model cherry; and

E. Petunia surfinia model ‘Dark violet’ grafted with Calibrachoa sp. ‘Chameleon’ model Purple.

6. Additional development process included:

6.1 Screening for additional Calibrachoa propagation material to be used as rootstock.

6.2 Improving the compatibility between the rootstock and the scion, to achieve the following parameters:

A. Uniform adhesion point—similar shoot diameters between rootstock and scion, without noticeable callus.

B. Short time adhesion, i.e. about 5 to 10 days from grafting to adhesion.

C. Suppression of continuous formation of side-shoots from the stem or sprouts from the callus.

D. Formation of a thickening and woody stem during the plant growth.

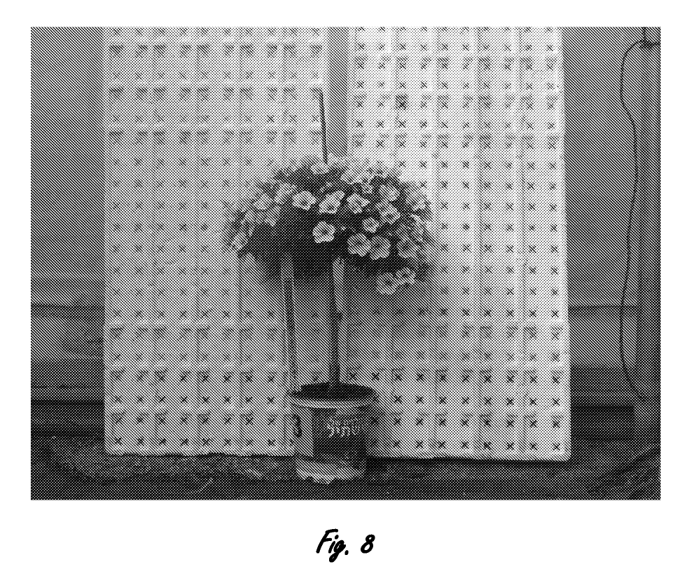

Reference is now made to FIG. 6 presenting a photographic illustration of grafting point breakdown of Calibrachoa sp. Chameleon pink grafted upon Petunia surfinia pink. It is shown in replicates 80, 90 and 100 that a weak adhesion and different rootstock and scion development lead to unequal diameter and physical sensitivity between the rootstock and scion.

6.3 Development of trailing and stabilizing means for supporting and shaping the grown rootstock or scion.

### Example 2

### An Exemplified Protocol for Grafting a Calibrachoa Scion onto a Selected Rootstock

One example of a method for producing a Calibrachoa grafted plant according to the present invention may encompass the following steps:

Step 1:

Selecting a plant, preferably of the Solanaceae family, suitable to be used as a compatible rootstock for grafting with plant of the Calibrachoa genus.

Step 2:

Growing the selected rootstock for a period of about 30 to about 60 days (dependent on the season) under predetermined agrotechnical conditions such as illumination, shading, temperature, irrigation and fertilization regimes, to achieve a rootstock with predetermined stem parameters needed for grafting (such as stem height, diameter, length between nodes etc.), to be engrafted with a Calibrachoa scion. In a specific embodiment, stem height of up to 0.5 m till the first bud is used.

It should be noted that the propagation material used for producing the rootstock may be selected from seeds, seedlings or cuttings. More specifically, the rootstock may be produced from young plants, rooted cuttings, unrooted cuttings, seedlings, grafted rooted cuttings, grafted unrooted cuttings or grafted unrooted seedlings. In the case of seeds the protocol is based on growing seedlings and using cuttings derived from the seedlings which are subjected to specific predetermined growth conditions adjusted to elongate the rootstock to the needed dimensions. The growth protocol for seeds as compared to cuttings is based on different lead time for each stage.

In a specific embodiment, rootstock plant cuttings are rooted in a planting tray under growth conditions optimal for producing erectly or upright sturdy herbaceous stem with a predetermined height suitable for grafting with the Calibrachoa scion.

Particularly, the selected rootstock is grown in predesigned conditions in order to be suitable for grafting with the Calibrachoa scion. The steps for growing the selected rootstock may encompass the following:

A. Adjusting the density of the grown rootstock plants (i.e. number of plants per area);

B. Selecting a suitable growth tray. The parameters for selecting such a suitable tray may include, in a non limiting manner size, shape of cells, material and density of the cells;

C. Applying controlled “Low-Radiation” regimes to produce elongated rootstock to the needed dimensions (i.e. for obtaining etiolated plants);

D. Controlling flowering timing, i.e. by affecting photoperiodic response of the plants;

E. Using trailing and stabilizing means to support the grown rootstock plants. Non limiting examples of such means include sticks and clips and other means and methods such as cut flowers, using a trailing net and/or using a plastic tray frame in order to support the grown rootstock.

F. Growing the selected rootstock at predetermined protocol, i.e. by controlling parameters such as illumination level, temperature, relative humidity, electrical conductivity, pH, irrigation intervals and fertilization solutions and regimes;

G. Optionally, applying plant protection treatments (e.g. pesticide-based approaches such as herbicides, insecticides and fungicides and biological pest control approaches, and a combination thereof);

I. Coordinating, regulating and manipulating properties of the plant propagation material to be used for rootstock production, including properties such as: root system development, i.e. the rate and conditions for development, size, functionality and rooting output, hypocotyl size, i.e. length; stem diameter and nodes number and length.

Step 3:

Growing a Calibrachoa species to be used as a scion suitable for grafting onto the selected rootstock. The Calibrachoa scion may be derived from young plants or propagation material or raw material comprising rooted cuttings, unrooted cuttings, seedlings, grafted rooted cuttings, grafted unrooted cuttings or grafted unrooted seedlings. In a specific embodiment, scion cuttings are grown under conditions adapted to adjust the scion stem diameter to be suitable for the rootstock diameter and grafting technique. The growth of the scion is manipulated such that the scion is suitable for grafting with the selected rootstock by growth characteristics such as growth staging and timing and stem diameter.

Step 4:

Grafting the Calibrachoa scion onto the manipulated rootstock at a predetermined height, by any suitable grafting technique or method. Optionally, using trailing and stabilizing means to support the grafted young plant. Non limiting examples of such trailing and stabilizing means include sticks, clips, flowers cutting, trailing net, plastic tray frame and any combination thereof.

Step 5:

Enhancing adhesion and hardening processes between the rootstock and scion tissues at the grafting area.

Step 6:

Planting the grafted Calibrachoa plant into a finalized or desired pot or growing container or planter and optionally using means to support the growth of the engrafted plant if required to provide the desired commercially valuable new engrafted Calibrachoa plant.

It is further within the scope that the grafting protocol of the present invention is used to produce grafted propagation material in the form young plants (such as grafted rooted cuttings and grafted seedlings) or any other raw material, such as unrooted grafted cuttings.

It is further within the scope to produce by the grafting protocol of the present invention high stem grafted Calibrachoa and high stem grafted propagation material in the form young plants (such as high stem grafted rooted cuttings and high stem grafted seedlings) or any other raw material, such as high stem unrooted grafted cuttings.

It is further within the scope that the grafting method may be any conventional or known grafting technique. In all methods employed, complementary ends of the scion and the rootstock are brought together to form a graft union. Callous tissue forms at the graft union as part of the normal healing process of the plant and serves as a conduit for water and nutrients between the scion and rootstock. Grafting involves the union of two independent plant parts into one plant. More specifically it may encompass any grafting technique which is suitable for grafting herbaceous plants, such as tomato plants. More particularly, the grafting method uses diagonal cutting with clips.

It is emphasized that the example described herein encompasses means and methods for grafting a scion of the Calibrachoa genus with any selected rootstock, which is found to be compatible with said Calibrachoa scion. Particularly, the example refers to means and methods for grafting a Calibrachoa scion with a rootstock of the Solanaceae family. More particularly, the rootstock used for grafting with the Calibrachoa scion may be a species of the Solanum, Capsicum, Petunia or Calibrachoa genus.

It is further emphasized that the exemplified method described herein encompasses the possibility of grafting two or more scions on one rootstock. In this embodiment, the two or more scions belong to different species within the same genus or different varieties of the same species. The different scions may have different characteristics such as inflorescence color, fruit shape and growing pattern. This enables the production of unique and surprising grafted plants and grafted propagation material having ornamental and horticultural commercially desirable combinations.

### Example 3

### Grafting a Calibrachoa Scion onto a Rootstock of the Petunia Genus

This example describes several embodiments showing Calibrachoa plants grafted upon rootstock of the Petunia genus and produced by the method of the present invention.

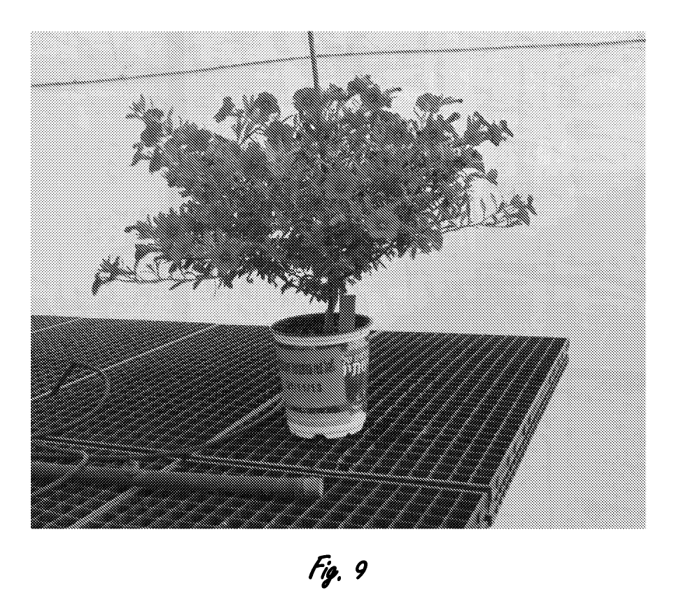

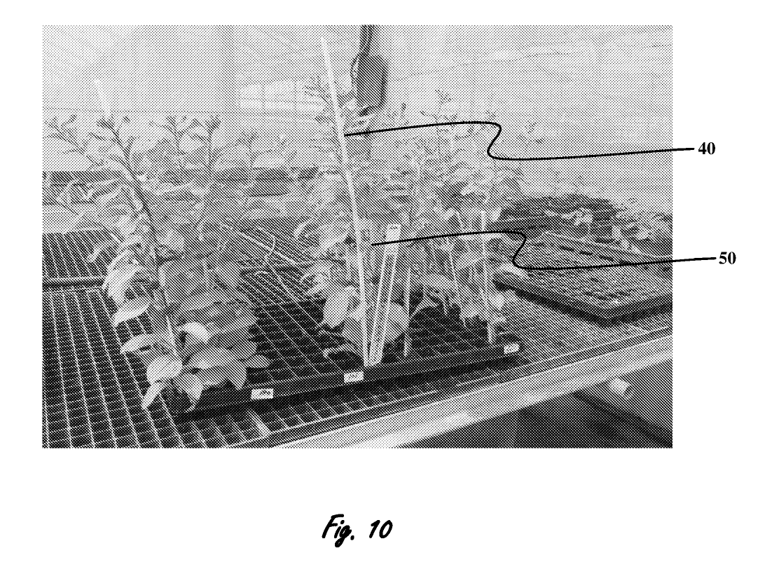

Reference is now made to FIGS. 7 and 8 presenting photographic illustrations of enhanced height engrafted Calibrachoa plants of various inflorescence colors. In this embodiment, a Calibrachoa scion is grown in predetermined growth characteristics to be compatible for engrafting upon a rootstock of the Petunia genus. According to a further embodiment, the Petunia plant is grown to be suitable as a rootstock having a stem height of about 20 cm, upon which the Calibrachoa scion is engrafted. In a specific embodiment, the Petunia rootstock and the Calibrachoa scion are grown under growth conditions designed so as to produce a stem diameter which is compatible between the scion and the rootstock.

FIG. 7 presents a photographic illustration of three high stem engrafted Calibrachoa plants comprising different Calibrachoa varieties grafted onto Petunia rootstocks of about 20 cm height. Each Calibrachoa variety has different inflorescence color. The scion used is Calibrachoa sp. Celebration®, from left to right: Neon rose, White improved and Yellow banana.

FIG. 8 presents a photographic illustration of a finished and shaped, and enhanced-height, engrafted Calibrachoa plant grown for at least two months under greenhouse conditions.

It is herein emphasized that the grafting of the Calibrachoa scion onto the rootstock can be done by any conventional grafting technique known in the art and more specifically by a grafting technique suitable for grafting herbaceous plants. Non limiting examples of grafting methods used in the present invention may include splice grafting, bud grafting, cleft grafting, side grafting, approach grafting, hole insertion grafting, one cotyledon grafting, whip grafting, stub grafting, four flap grafting, awl grafting, veneer grafting and any other grafting technique suitable for grafting herbaceous plant and any combination thereof.

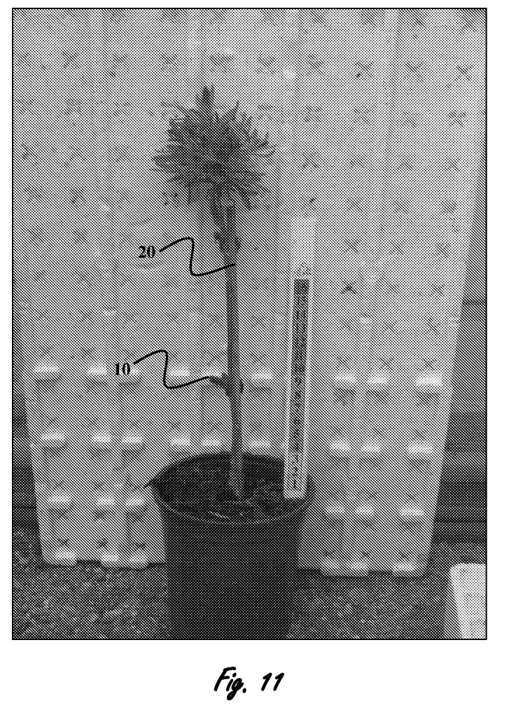

Reference is now made to FIG. 9, presenting a photographic illustration of a short-stock engrafted Calibrachoa plant. In this embodiment, a Calibrachoa scion is engrafted onto a petunia rootstock at a height of about 10 cm.

Reference is now made to FIG. 10 presenting a photographic illustration of cultivated Petunia plants grown as rootstock, to be grafted with the Calibrachoa scion. It is herein acknowledged that both Petunia and Calibrachoa genus belong to the Solanceae family, and species of both genera were characterized. Most of the Petunia species are herbaceous and annual. Most Calibrachoa species are predominantly sub-arbustive and perennial. In this embodiment a plant of the Petunia genus is grown as a rootstock to be grafted at a height of at least 16 cm with a Calibrachoa scion. As can be seen in this figure, special sticks 40 (of about 20 cm) and clips 50 that attach the rootstock to the stick have been used to support the rootstock growth. Furthermore, methods and means such as cut flowers, trailing net and a plastic tray frame may be used in order to support the grown rootstock.

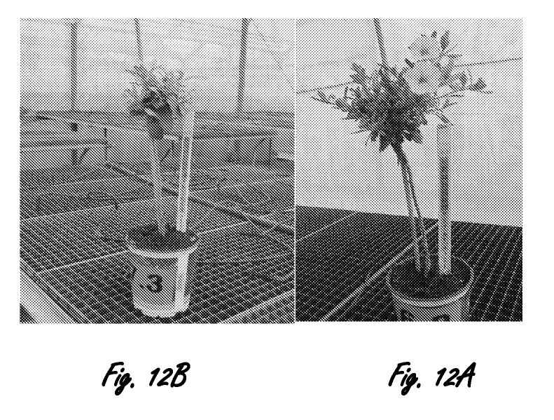

Reference is now made to FIG. 11 presenting a photographic illustration of an enhanced height Calibrachoa grafted plant. In this embodiment, a Calibrachoa scion is engrafted upon a Petunia rootstock at a height of about 20 cm. It is herein acknowledged that plant species of the Calobrachoa genus naturally have a sub-arbustive or shrubby growth pattern and a week root system, which may also be susceptible to heat, salt and pH. This embodiment of the present invention demonstrates one advantage of the present invention, enabling the achievement of elevated or enhanced height Calibrachoa plant with a strong stem and root system. The elevated Calibrachoa plant demonstrated in FIG. 11 is supported and stabilized by clips 10 and by rod 20.

Reference is now made to FIG. 12 presenting a photographic illustration of enhanced height engrafted Calibrachoa plant before (A) after (B) pruning. In this embodiment, a Calibrachoa scion was engrafted onto a Petunia rootstock.

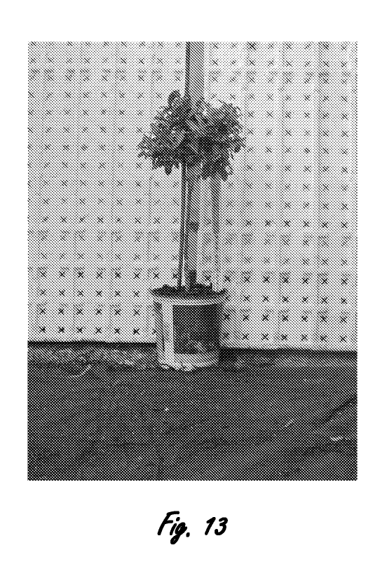

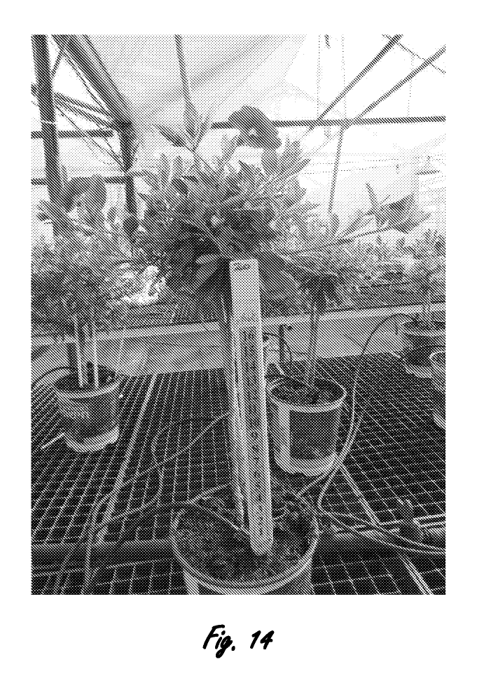

Reference is now made to FIG. 13 presenting a photographic illustration of a mature engrafted Calibrachoa plant after pruning. The Calibrachoa scion is engrafted onto a Petunia rootstock at a height of about 16 cm or more. According to certain aspects, the pruning encourages renewal of the growth of the scion stems, leaves and flowers.

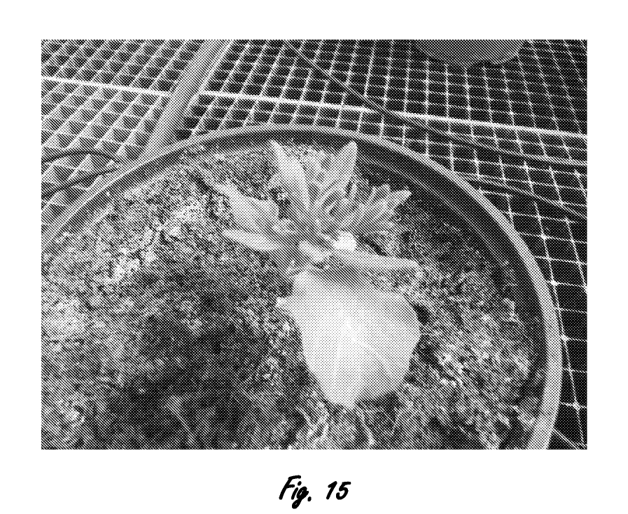

Reference is now made to FIG. 14 presenting a photographic illustration of enhanced height Calibrachoa grafted plant, 2 to 3 weeks after transplantation to a pot. This embodiment shows a grafted plant, 2-3 weeks after it was transplanted to a pot and before pruning of the plant in order to shape its final presentation.

### Example 4

### Grafting a Calibrachoa Scion onto a Rootstock of the Solanum Genus

This example describes several embodiments showing Calibrachoa plants grafted upon rootstock of the Solanum genus, particularly of the S. melongena species and produced by the method of the present invention.

Reference is now made to FIG. 15 presenting a photographic illustration of a Calibrachoa scion grafted upon a rootstock of the S. melongena species as an embodiment of the present invention. It is shown in this figure that a grafted Calibrachoa plant comprising a Calibrachoa scion grafted upon S. melongena rootstock also known as eggplant, aubergine, melongene, garden egg, guinea squash or asbrinjal, is successfully produced due to the compatibility between the chosen rootstock and scion and the proper growth and development of the rootstock which was designed according to the grafting protocol of the present invention.

1.-90. (canceled)

91. A grafted Calibrachoa plant, wherein said plant comprises a rootstock engrafted with a Calibrachoa scion.

92. The plant according to claim 91, wherein said rootstock is compatible with said Calibrachoa scion by at least one compatible characteristic.

93. The plant according to claim 91, wherein said plant is derived from propagation material selected from the group consisting of young plants and unrooted raw material.

94. The plant according to claim 93, wherein at least one of the following holds true:
a. said unrooted raw material is selected from the group consisting of unrooted cuttings and unrooted grafted cuttings;
b. said unrooted raw material is adapted for insertion and growth in plug trays; and
c. said young plants are selected from the group consisting of rooted cuttings, seedlings, grafted rooted cuttings, and grafted seedlings.

95. A finished plant derived from the propagation material of claim 93.

96. The plant according to claim 91, wherein at least one of the following holds true:
a. said rootstock and/or said scion is derived from propagation material selected from the group consisting of seeds, rooted cuttings, unrooted cuttings, seedlings, grafted rooted cuttings, grafted unrooted cuttings and grafted unrooted seedlings;
b. said rootstock belongs to the Solanaceae family;
c. said scion is of the Calibrachoa genus;
d. said scion is engrafted at a height of up to 50 cm on said rootstock;
e. said scion is engrafted at a height of at least 10 cm on said rootstock;
f. said scion is characterized by at least one characteristic selected from the group consisting of dense flowering, unique flower color, low to moderate incompatibility with the rootstock, at least 50% output of developed plants, and any combination thereof;
g. said rootstock is engrafted with at least two different scions said scions belong to different varieties and are compatible with said rootstock;
h. said plant is planted into a final commercial planter or pot or any other growing container;
i. said plant is a high stem grafted Calibrachoa;
j. said rootstock has desirable agronomical and/or horticultural characteristics selected from the group consisting of high rooting potential, high rooting quality, developed root system, callus formation, minimal sprouts from the callus, minimal side-shoots development from the stem, thick stem, long internodes, robustness, sparse flowering, production of a straight trunk, woodiness, strong root system, enhanced stem height, plant vigor, stress tolerance, disease resistance, resistance to pathogens, resistance to insect infestation, resistance to abiotic stress, a rooting potential of at least 80%, sprout differentiation from callus lower than 80%, side shoots from the stem lower than 90%, average stem thickness of at least 5 mm, average internode length of at least 10 mm and any combination thereof; and
k. said engrafted Calibrachoa plant has desirable ornamental and/or agronomical and/or horticultural characteristics relative to a non-grafted Calibrachoa plant selected from the group consisting of: enhanced height or high stem plant, uniform adhesion point, woodiness, similar shoot diameter between the rootstock and the scion, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof.

97. The plant according to claim 91, wherein said rootstock is selected from the group consisting of Cestroideae, Goetzeoideae, Nicotianoideae, Petunioideae, Schizanthoideae, Schwenckioideae and Solanoideae subfamily.

98. The plant according to claim 91, wherein said rootstock is selected from the group consisting of Solanum, Capsicum, Petunia and Calibrachoa genus.

99. The plant according to claim 91, wherein said rootstock is selected from the group consisting of Petunia alpicola, P. altiplana, P. axillaris, P. bajeensis, P. bonjardinensis, P. exserta, P. guarapuavensis, P. helianthemoides, P. humifusa, P. inflate, P. integrifolia, P. interior, P. ledifolia, P. littoralis, P. mantiqueirensis, P. occidentalis, P. parviflora, P. patagonica, P. pubescens, P. reitzii, P. riograndensis, P. saxicola, P. scheideana, P. variabilis, P. villadiana, P.×atkinsiana, P. hybrid, Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia, Calibrachoa×hybrid, Petchoa, lycopersicum, tuberosum and melongena species.

100. The plant according to claim 91, wherein said scion is selected from the group consisting of Calibrachoa caesia, Calibrachoa calycina, Calibrachoa dusenii, Calibrachoa eglandulata, Calibrachoa elegans, Calibrachoa ericaefolia, Calibrachoa excellens, Calibrachoa hassleriana, Calibrachoa heterophylla, Calibrachoa humilis, Calibrachoa linearis, Calibrachoa parviflora, Calibrachoa pygmaea, Calibrachoa rupestris, Calibrachoa sellowiana, Calibrachoa spathulata and Calibrachoa thymifolia species.

101. The plant according to claim 92, wherein said at least one compatible characteristic with said scion is selected from the group consisting of: similar stem diameter, similar shoot diameter, similar growth pattern, taxonomical similarity, genetic similarity, anatomical similarity, formation of uniform adhesion point between said rootstock and said scion, absence of noticeable callus, suppressed formation of side shoots from the stem, suppressed formation of sprouts from the callus, a thick and woody stem, normal development of said scion similar or enhanced relative to a corresponding none grafted control plant, and any combination thereof.

102. The plant of claim 96, wherein at least one of the following holds true:
a. the stress tolerance comprises an abiotic stress tolerance selected from the group consisting of cold tolerance, high temperature tolerance, drought tolerance, salt tolerance, pH tolerance and any combination thereof;
b. the stress tolerance comprises a biotic stress tolerance selected from the group consisting of a disease resistance, an insect resistance, a nematode resistance, improved resistance to soil borne pathogens and any combination thereof;
c. said different scions have at least one different characteristic selected from the group consisting of: inflorescence color, fruit shape, growth pattern and any combination thereof; and
d. said high stem grafted Calibrachoa plant is derived from propagation material selected from the group consisting of high stem grafted young plants and unrooted high stem grafted raw material.

103. The plant according to claim 102, wherein at least one of the following holds true:
a. said high stem young plants are selected from the group consisting of rooted cuttings, seedlings, grafted rooted cuttings, and grafted seedlings;
b. said high stem unrooted raw material is selected from the group consisting of unrooted cuttings and unrooted grafted cuttings; and
c. said high stem unrooted raw material is adapted for perpendicular stability and/or sturdiness when planted in a plug tray.

104. A high stem grafted finished plant derived from the propagation material of claim 102.

105. The cuttings or unrooted cuttings, grafted cuttings or grafted unrooted cuttings of the high stem grafted Calibrachoa according to claim 96.

106. Any part of the plant according to claim 91, said plant part is selected from the group consisting of rooted cuttings, unrooted cuttings, grafted rooted cuttings, unrooted grafted cuttings, flowers and any other vegetative and reproductive part of the plant.

107. Grafted propagation material of the plant of claim 91, wherein said propagation material is selected from the group consisting of grafted young plants, grafted rooted cuttings, grafted unrooted cuttings, grafted seedlings, high stem grafted young plants and high stem grafted unrooted cuttings.

108. A method for producing a Calibrachoa engrafted plant comprising steps of:
a. providing a scion of the genus Calibrachoa;
b. conditioning a rootstock for compatibility with said Calibrachoa scion;
c. grafting said scion onto said rootstock, thereby producing a grafted Calibrachoa plant.

109. The method according to claim 108, wherein said step of conditioning, further comprises at least one step of:
a. growing said rootstock to have desirable agronomical and/or horticultural characteristics selected from the group consisting of high rooting potential, high rooting quality, minimal side-shoots development from the stem, thick stem, long internodes, robustness, sparse flowering, producing a straight trunk, woodiness, strong root system, enhanced stem height, plant vigor, stress tolerance, disease resistance, resistance to pathogens, resistance to insect infestation, resistance to abiotic stress, a rooting potential of at least 80%, sprout differentiation from callus lower than 80%, side shoots from the stem lower than 90%, average stem thickness of at least 5 mm, average internodes elongation of at least 10 mm, and any combination thereof; and
b. growing said rootstock grafted upon said scion and obtaining characteristics selected from the group consisting of a uniform adhesion point between said rootstock and said scion, similar shoot diameters between said rootstock and said scion, absence of noticeable callus, suppressed formation of side shoots from the stem, suppressed formation of sprouts from the callus and any combination thereof.

110. The method according to claim 108, further comprises at least one step of:
a. selecting a scion having at least one characteristic selected from the group consisting of dense flowering, unique flower colors, low to moderate incompatibility with the rootstock, at least 50% output of developed plants, and any combination thereof;
b. producing and growing propagation material selected from the group consisting of young plants and unrooted raw material at predetermined growth conditions so as to provide said Calibrachoa grafted plant;
c. growing propagation material or young plants or raw material selected from the group consisting of seeds, rooted cuttings, unrooted cuttings, seedlings, grafted rooted cuttings, grafted unrooted cuttings and grafted seedlings of said rootstock plant at predetermined growth conditions so as to be suitable for grafting with said scion;
d. growing propagation material or young plants or raw material selected from the group consisting of seeds, rooted cuttings, unrooted cuttings, seedlings, grafted rooted cuttings, grafted unrooted cuttings and grafted seedlings of said scion at predetermined growth conditions so as to be suitable for grafting with said rootstock;
e. adjusting the density of the grown rootstock plants by controlling the number of plants per area;
f. selecting a suitable growth tray by parameters selected from the group consisting of size, shape of cells, material, density and any combination thereof;
g. growing said rootstock under predetermined fertigation and shading regimes;
h. applying controlled Low-Radiation regimes for obtaining etiolated rootstock plants;
i. controlling the flowering timing of the grown rootstock plants by affecting photoperiodic response of the plants;
j. using trailing and stabilizing means for supporting and shaping the grown rootstock;
k. growing the selected rootstock plants at a predetermined protocol by controlling parameters selected from the group consisting of temperature, relative humidity, electrical conductivity, illuminating level, shading level, pH, irrigation intervals, fertilization solutions and regimes and any combination thereof;
l. applying plant protection treatments selected from the group consisting of: pesticide-based treatment, herbicides treatment, insecticides treatment, fungicides treatment, biological pest control treatment and any combination thereof;
m. coordinating at least one property of the plant propagation material to be grown for rootstock production, said at least one property is selected from the group consisting of root system development, hypocotyl size, hypocotyl length, stem diameter, nodes number, internode length and any combination thereof;
n. enhancing adhesion between said scion and said rootstock to form a graft union;
o. growing said rootstock to a predetermined height of up to 50 cm and to a predetermined stem diameter;
p. growing said rootstock to a predetermined height of at least 10 cm;
q. manipulating and coordinating the growth of said rootstock plant so as to achieve growth characteristics suitable for grafting with said scion, said growth characteristics are selected from the group consisting of: root system development, hypocotyl size, hypocotyl length, stem diameter, nodes number, node length, stem diameter, stem height, growth pattern, flowering time and any combination thereof;
r. grafting said scion onto said rootstock by a grafting technique selected from the group consisting of: splice grafting, bud grafting, cleft grafting, side grafting, approach grafting, hole insertion grafting, one cotyledon grafting, whip grafting, stub grafting, four flap grafting, awl grafting, veneer grafting, any other grafting technique suitable for grafting herbaceous plant and any combination thereof;
s. planting said grafted plant into a final commercial planter or pot or any other growing container;
t. producing an engrafted Calibrachoa plant with desirable ornamental and/or horticultural characteristics relative to non-grafted Calibrachoa plant selected from the group consisting of: enhanced height or high stem plant, strong root system, tolerance to biotic stress, tolerance to abiotic stress, uniformity, reduced leaf yellowing, robust growth, enhanced inflorescence yield, enhanced yield of plant propagation material such as cuttings, enhanced aerial yield, combination of more than one Calibrachoa species or variety on one plant and any combination thereof;
u. producing a high stem grafted Calibrachoa; and
v. engrafting said rootstock with at least two different scions, said scions belong to different varieties and are compatible with said rootstock.

111. The method according to claim 110, further comprises at least one step of:
a. selecting said young plants from the group consisting of rooted cuttings, seedlings, grafted rooted cuttings, and grafted seedlings;
b. selecting said unrooted raw material from the group consisting of unrooted cuttings and unrooted grafted cuttings;
c. growing said propagation material so as to produce a finished grafted Calibrachoa plant;
d. growing said unrooted raw material in plug trays;
e. planting said grafted plant into a final commercial planter or pot or any other growing container having a volume of 1 liter and less;
f. planting said grafted plant into a final commercial planter or pot or any other growing container having a volume of 1 liter and more;
g. growing the rootstock to a predetermined height of at least about 10 cm and predetermined stem diameter and grafting the Calibrachoa scion upon said rootstock at said predetermined height;
h. producing and growing propagation material selected from the group consisting of high stem young plants and unrooted high stem raw material at predetermined growth conditions so as to provide said Calibrachoa high stem grafted plant; and
i. providing said different scions having at least one different characteristic selected from the group consisting of: inflorescence color, fruit shape, growth pattern and any combination thereof.

112. The method according to claim 111, further comprises at least one step of
a. selecting said high stem young plants from the group consisting of rooted high stem cuttings, high stem seedlings, grafted high stem rooted cuttings, and grafted high stem seedlings;
b. selecting said high stem unrooted raw material from the group consisting of unrooted high stem cuttings and unrooted high stem grafted cuttings; and
c. growing said propagation material so as to produce a finished high stem grafted Calibrachoa plant.

113. Any part of a plant produced by the method according to claim 108, said plant part is selected from the group consisting of rooted cuttings, unrooted cuttings, grafted rooted cuttings, unrooted grafted cuttings, flowers and any other vegetative and reproductive part of the plant.

114. Grafted propagation material of a plant produced by the method of claim 108, wherein said propagation material is selected from the group consisting of grafted young plants, grafted rooted cuttings, grafted unrooted cuttings, grafted seedlings, high stem grafted young plants and high stem grafted unrooted cuttings.
